(19) United States
(12) Patent Application Publication
(10) Pub. No.: US 2003/0055107 A1
(43) Pub. Date: Mar. 20, 2003

(54) Forms of pharmaceutically active agents and method for manufacture thereof

(76) Inventors: Xinmin Samuel Xu, Belle Mead, NJ (US); David F. Erkoboni, Pennington, NJ (US); David B. Lebo, Warminster, PA (US)

Correspondence Address: Donald O. Nickey, Cardinal Health, Inc., 7000 Cardinal Place, Dublin, OH, 43017, US

(21) Appl. No.: 10/107,759

(22) Filed: Mar. 27, 2002

Related U.S. Application Data
(60) Provisional application No. 60/279,210, filed on Mar. 27, 2001.
(60) Provisional application No. 60/279,489, filed on Mar. 28, 2001.

(51) Int. Cl. A61K 31/19; A61K 31/13; A61K 31/16

(52) U.S. Cl. 514/557; 514/579

(57) ABSTRACT

A pharmaceutical composition comprising a pharmaceutically active agent and a salt of said pharmaceutically active agent with the proviso that said composition does not contain hydrolyzed cellulose, wherein said pharmaceutical active agent is a weak acid or weak base. The present invention is also directed to a new ibuprofen form having, when potassium is present in said form as a counter ion, an IR peak at 1706 cm⁻¹ shifted and broadened in absorbance relative to racemic ibuprofen free acid, the form's characteristic absorbance profile from 1000 to 650 cm⁻¹ and broadened in absorbance relative to racemic ibuprofen free acid, the form's characteristic absorbance profile from 1000 to 650 cm⁻¹ and D-spacings of 21.1, 7.1, and 3.4 Å by X-ray diffraction.

### RELATED APPLICATIONS

This application is based upon and claims priority to U.S. Provisional Application Serial No. 60/279,210 filed Mar. 27, 2001 and U.S. Provisional Application Serial No. 60/279,489 filed Mar. 28, 2001.

### FIELD OF THE INVENTION

The present invention relates to new pharmaceutical compositions, new forms of pharmaceutically active agents and to the manufacture thereof. The new forms of the pharmaceutically active agents have improved aqueous dissolution as compared to the pharmaceutically active agents and, as a result, provide improved bioavailability of the pharmaceutically active agents. The present invention also relates to a process for improving the absorption profile of pharmaceutically active agents.

### BACKGROUND OF THE INVENTION

A common and important goal throughout the pharmaceutical industry is to improve the dissolution and thereby the rate of absorption and bioavailability of all pharmaceutically active agents. Certain relatively water-insoluble pharmaceutically active agents are difficult to handle and present additional challenges to the formulator due to their insolubility as the free acid or base form and the hygroscopic nature of the more soluble salt form. Ibuprofen, a well known member of the propionic acid group of nonsteroidal anti-inflammatory drugs (NSAIDS), is an example of such a pharmaceutically active agent and is widely regarded as a difficult agent to formulate for immediate release. It is a racemic mixture, has a melting point of 75° to 77° C. and is practically insoluble in water (<0.1 mg/mL).

The potassium salt of ibuprofen is highly soluble however is difficult to formulate because of its hygroscopic nature. Ibuprofen, as used herein, refers to the racemic free acid of ibuprofen.

Several techniques have been used with varying degrees of success to handle such difficult-to-process pharmaceuticals and provide them in acceptable dosage forms. It is conventional to dry hygroscopic pharmaceutically active agents prior to use and to process them together with various tableting additives under low humidity conditions and to then compress the resulting blend into tablets which can be coated or placed into humidity-resistant packaging. Moisture pickup by such hygroscopic pharmaceutically active agents can cause recrystallization, crystal growth and/or bridging between the crystals of the pharmaceutically active agent (thereby retarding the absorption profile of these relatively soluble pharmaceutically active agents). Hygroscopic materials are known to be problematic during the tableting operation. Tablets containing hygroscopic materials can also be difficult to coat with aqueous systems.

The pharmaceutical industry continues to search for new ways to improve the dissolution, rate of absorption and bioavailability of all pharmaceutically active agents, particularly, those pharmaceutically active agents which are relatively water insoluble. There is also the need to increase the processability and physical stability of hygroscopic pharmaceutically active agents.

### SUMMARY OF THE INVENTION

The present invention is directed to a pharmaceutical composition comprising a pharmaceutically active agent and a salt of said pharmaceutically active agent with the proviso that said composition does not contain hydrolyzed cellulose, wherein said pharmaceutically active agent is a weak acid or weak base.

The present invention is also directed to a new ibuprofen form having, when potassium is present in said form as a cation, an IR peak at 1706 cm⁻¹ and D-spacings of 21.1, 7. 1, and 3.4 Å by X-ray diffraction. The new ibuprofen form of the present invention can be formulated into a solid dosage form and combined with other pharmaceutically active agents, as well as pharmaceutically acceptable excipients.

### DESCRIPTION OF THE DRAWINGS

FIGS. 1-4 are the FTIR (ATR) spectra for ibuprofen, potassium ibuprofen, the new ibuprofen form of the present invention wherein the cation for the new form is potassium and a spray dried formulation of the new ibuprofen form wherein the cation for the new form is potassium, respectively.

FIG. 5 is an overlay of FTIR spectra for ibuprofen, potassium ibuprofen, mixtures thereof, and new ibuprofen form of the present invention wherein the cation for the new form is potassium.

FIGS. 6-8 are the Raman spectra for ibuprofen, potassium ibuprofen and the new ibuprofen form of the invention wherein the cation for the new form is potassium.

FIG. 9 is a phase diagram for ibuprofen, potassium ibuprofen and the new ibuprofen form (referred to as “Compound” in the diagram), wherein the cation in the new form is potassium. The phase diagram shows the melting points from DSC data of varying molar mixtures of ibuprofen, potassium ibuprofen and the new ibuprofen form wherein the new ibuprofen form is shown at the 50:50 molar ratio having no excess of ibuprofen or potassium ibuprofen.

FIGS. 10-12 are the X-ray diffraction patterns for ibuprofen, potassium ibuprofen and the new ibuprofen form of the invention wherein the cation in the new form is potassium. See Tables 1 (ibuprofen), 2 (potassium ibuprofen) and 3 (new ibuprofen form) below.

FIG. 13 is a 3-D X-ray diffraction pattern overlay for ibuprofen, potassium ibuprofen, the new ibuprofen form of the invention wherein the cation of the new form is potassium, and mixtures thereof.

FIGS. 14-16 are SEM images of ibuprofen, potassium ibuprofen and the new ibuprofen form of the invention wherein the cation of the new form is potassium.

FIG. 17 is a DSC thermalgram for the phase diagram of FIG. 9 for potassium ibuprofen.

FIG. 18 is a DSC thermalgram for the phase diagram of FIG. 9 showing the new ibuprofen form at 118.9° C. in admixture with an excess of potassium ibuprofen made at the stated molar mixture of ibuprofen to potassium ibuprofen.

FIG. 19 is a DSC thermalgram for the phase diagram of FIG. 9 showing the new ibuprofen form at 118.99° C. in admixture with an excess of potassium ibuprofen made at the stated molar mixture of ibuprofen to potassium ibuprofen.

FIG. 20 is a DSC thermalgram for the phase diagram of FIG. 9 showing the melting point for a eutectic composition at 117.97° C. containing the new ibuprofen form (wherein the cation of the new form is potassium) wherein the eutectic composition was made at the stated molar mixture of ibuprofen to potassium ibuprofen. The melting point is depressed due to a eutectic point between the new ibuprofen form and potassium ibuprofen.

FIG. 21 is a DSC thermalgram for the phase diagram of FIG. 9 showing the new ibuprofen form at 121.34° C. as made in Example 1.

FIG. 22 is a DSC thermalgram for the phase diagram of FIG. 9 showing the melting point for a eutectic composition at 59.47° C. containing the new ibuprofen form (wherein the cation of the new form is potassium) wherein the eutectic composition was made at the stated molar mixture of ibuprofen to potassium ibuprofen. The melting point is depressed due to a eutectic point between the new ibuprofen form and ibuprofen.

FIG. 23 is a DSC thermalgram for the phase diagram of FIG. 9 showing the melting point onset at 58.79° C. for a mixture containing the new ibuprofen form (wherein the cation of the new form is potassium) and an excess of ibuprofen made at the stated molar mixture of ibuprofen to potassium ibuprofen.

FIG. 24 is a DSC thermalgram for the phase diagram of FIG. 9 showing the melting point onset at 58.67° C. for a mixture containing the new ibuprofen form (wherein the cation of the new form is potassium) and an excess of ibuprofen made at the stated molar mixture of ibuprofen to potassium ibuprofen.

FIG. 25 is a DSC thermalgram for the phase diagram of FIG. 9 showing two melting point onsets at 67.05° C. for a mixture containing the new ibuprofen form (wherein the cation of the new form is potassium) and an excess of ibuprofen made at the stated molar mixture of ibuprofen to potassium ibuprofen.

FIG. 26 is a DSC thermalgram for the phase diagram of FIG. 9 for ibuprofen 38 (BASF).

FIG. 27 is UV spectra showing identical dissolution for ibuprofen (“ibuprofen free acid”) and the new ibuprofen form (“ibuprofen form”) of the present invention, wherein the cation is potassium.

FIG. 28 is a single heat DSC thermalgram for a 50:50 mixture of ibuprofen/potassium ibuprofen showing formation of the new ibuprofen form at 120.27° C. and the melting of ibuprofen at 56.7° C.

FIG. 29 is a DSC thermalgram for a 50:50 mixture of ibuprofen/potassium ibuprofen heated only to 100° C. and then cooled showing a melting point of ibuprofen at 56.4° C.

FIG. 30 is a DSC thermalgram for a 50:50 mixture of ibuprofen/potassium ibuprofen heated to 100° C. and then cooled as in FIG. 29 followed by a second heating step showing the formation of the new ibuprofen form at 120.27° C. and no ibuprofen as compared with the single heating step in FIG. 29 or the single heating step of FIG. 28.

FIG. 31 is the X-ray Diffraction for a formulated product of the new ibuprofen form of the invention made in accordance with Example 5. See Table 4 below.

FIG. 32 is the dynamic water vapor absorption curve for the new ibuprofen form of the invention wherein the cation is potassium.

FIG. 33 is the dynamic water vapor absorption curve for potassium ibuprofen.

FIGS. 34-35 are the dynamic water vapor absorption curves for the new ibuprofen form of the invention (FIG. 34) and potassium ibuprofen (FIG. 35). See Example 9.

FIG. 36 shows the solubility of ibuprofen. Ibuprofen as shown in this Figure demonstrates an increased solubility at higher pH. The new form will have improved dissolution when compared to ibuprofen because the pH of the local environment around the new form would be higher than that for ibuprofen.

FIG. 37 is an X-ray diffraction pattern for ibuprofen and potassium ibuprofen as in Example 7. Throughout the Figures, reference may appear to a 50/50 ibuprofen to potassium ibuprofen compound or 50-50 compound. It should be understood that such is the new ibuprofen form of this invention.

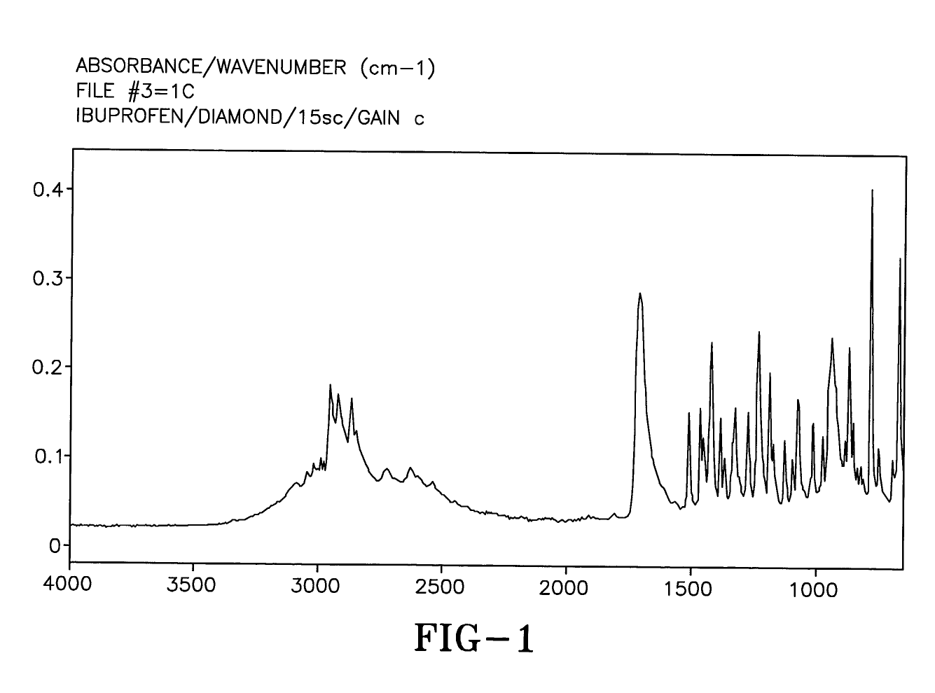

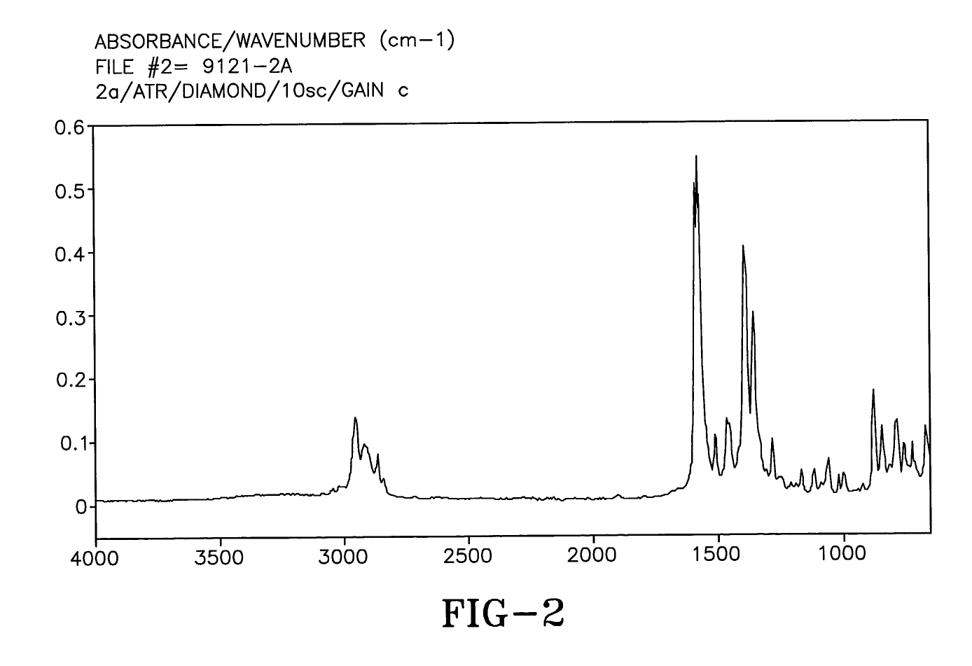

### DETAILED DESCRIPTION OF THE INVENTION

The present invention is directed to a pharmaceutical composition comprising a pharmaceutically active agent and a salt of said pharmaceutically active agent with the proviso that said composition does not contain hydrolyzed cellulose, wherein said pharmaceutically active agent is a weak acid or weak base. This pharmaceutical composition can be in at least one solid, non-crystalline state, at least one solid, crystalline state, at least one liquid crystalline form, a gel form or at least one aqueous based medium. The new compositions may be a homogenous or heterogeneous mixture, and eutectic system of pharmaceutically active agents and a salt of said active agent.

The pharmaceutical composition of the present invention forms a buffer which allows it to act differently than a pharmaceutical composition containing either the free form of the active agent or its salt alone. Thus, in an aqueous medium such as that in the gastric environment, a drug delivery platform using the pharmaceutical composition of the present invention may reduce gastric irritation and thereby reduce the associated toxicity of the drug.

Other pharmaceutically acceptable excipients, pharmaceutical active agents, salt forms thereof, can be contained in the pharmaceutical composition of the invention.

The present invention is directed to new forms of pharmaceutically active agents prepared by subjecting a mixture of such pharmaceutically active agents and their salts to certain conditions that create the new forms. Such methods comprise: 1) reacting a portion of a pharmaceutically active agent with an acid or base capable of reacting with such active agent followed by drying, 2) reacting a portion of a pharmaceutically active agent in molten form with an acid or base capable of reacting with such molten active agent or 3) processing the pharmaceutically active agent with the salt of its conjugate acid or conjugate base in the presence of heat, pressure, shear and/or water.

As stated above, these new forms have improved dissolution when compared with the pharmaceutically active agents. This improved dissolution results in a soluble pharmaceutically active agent having more rapid absorption and improved bioavailability. Without intending to be limited by any particular theory, it is believed that the new form obtained by the reaction between the acid or base and the pharmaceutically active agents under the conditions set forth herein results in a form of the pharmaceutically active agent which has a faster dissolution than the pharmaceutically active agent itself. This form is believed to have improved properties compared to both the pharmaceutically active agent or the salt of its conjugate acid or base. In the case of a poorly soluble pharmaceutical active, the form is expected to have increased solubility while the moisture uptake of the form is expected to be reduced compared to the pure salt of its conjugate acid or base of the pharmaceutical active.

The new forms of the present invention can be used in immediate release formulations, sustained-release formulations, controlled release formulations, and the like.

The pharmaceutically active agents that may be reacted with an acid or base in accordance with the present invention (to obtain the new form) may be, for example, any pharmaceutically active agent capable of forming a crystal structure or amorphous state distinct from either the pharmaceutically active agent or the salt of its conjugate acid or base. Such pharmaceutically active agents may include weak acids. Examples of weak acids include Nonsteroidal Anti-Inflammatory Agents (NSAIDS) such as ibuprofen, naproxen, ketoprofen and like compounds; barbiturates such a butalbital, phenobarbital, and secobarbital; penicillins and cephalosporins such as penicillin G, ampicillin, cefuroxime, cefazolin and like compounds; and anti-lipidemic drugs such as gembifrozil and anticonvulsants such as valproic acid. Such pharmaceutically active agents may include weak bases. Examples include antihistamines such as terfenadine, astemizole, diphenhydramine, chlorpheniramine maleate, brompheniramine maleate, triprolidone, loratadine, desloratadine; anti-depressants such as fluoxetine, imipramine, nortryptyline, and vanlafaxine; anti-psychotics such as chlorpromazine, clozapine, and haloperidol; anti-hypertensives such as antenolol, propranolol, and metoprolol; analgesics such as codeine, propoxyphene, hydrocodone; and decongestants such as phenylpropanolamine, phenylephrine hydrochloride, and pseudoephedrine hydrochloride.

Any acid or base capable of reacting with the pharmaceutically active agent and/or the salt of its conjugate acid or base may be used in the present invention. Examples of such a base include, but are not limited to, metal hydroxides such as potassium hydroxide, sodium hydroxide, calcium hydroxide and magnesium hydroxide as well as ammonium and quaternary ammonium hydroxides. Examples of such an acid include but are not limited to, hydrochloric acid, hydrobromic acid, sulfuric acid, phosphoric acid, citric acid, succinic acid, gluconic acid, glycolic acid, fumaric acid, tartaric acid, maleic acid and the like. Others include zwitterionic agents such as amino acids including argine, guanidine and lysine.

In another embodiment of the present invention, the pharmaceutically active agent and its conjugate salt may be processed together directly in the presence of heat, shear and/or water to obtain the new form having a distinct crystal structure.

The first process of the present invention involves the steps of reacting the acid or base in aqueous medium with the pharmaceutically active agent followed by drying.

The reaction may be facilitated by agitation. Typical drying conditions depend upon the pharmaceutically active agent. Drying must be done under temperature and pressure conditions which avoid precipitation of the pharmaceutical active agent. Importantly, drying must be done under conditions that maintain the active agent and its salt in intimate contact. It is believed that this intimate contact at least partially facilitates the conditions necessary to create the new form out of the mixture of the active agent and its salt. The temperature of the dispersion should not exceed that temperature at which the new compound formed is stable. Drying can be done by any conventional drying technique such as, for example, vacuum drying, solvent drying, freeze-drying, flash drying, and spray drying. In the case of the ibuprofen form of the present invention wherein the cation is potassium, drying is required in order to obtain the new ibuprofen form having a stoichiometry of 2 moles of ibuprofen to 1 mole of potassium. Typical drying conditions employed are from 25° C. to 75° C. in a vacuum oven. Addition of the ibuprofen form of the present invention wherein the cation is potassium to water results in immediate dissolution to provide ibuprofen.

The second process of the present invention involves the step of reacting the acid or base with the pharmaceutically active agent in its molten state. The acid or base may be added as a liquid or in solid form. The molten state of the pharmaceutically active agent will be that temperature and/or pressure at which the solid form of the pharmaceutically active agent forms a liquid and mixing can occur. The new form crystallizes when the temperature is lowered sufficiently for nucleation and growth to occur. The ibuprofen form of the present invention typically is prepared by melting ibuprofen at 100° C. and adding potassium hydroxide while stirring. Nucleation is observed with cooling below about 65° C. yielding the ibuprofen form having a stoichiometry of 2 moles of ibuprofen to one mole of potassium. While not bound by theory, it is believed that cooling below the melting point of ibuprofen nucleates the formation of the ibuprofen form.

The third process of the present invention involves the steps of processing the pharmaceutically active agents and the salt of its conjugate acid or base in the presence of heat, pressure, shear and/or moisture.

Regardless of the process employed, the highest yield of the ibuprofen form of the present invention is obtained when providing reagents in the stoichiometric ratio of the compound which is formed. For the ibuprofen form of the present invention wherein the cation is potassium, the stoichiometry is 2 moles of ibuprofen to 1 mole of potassium ion.

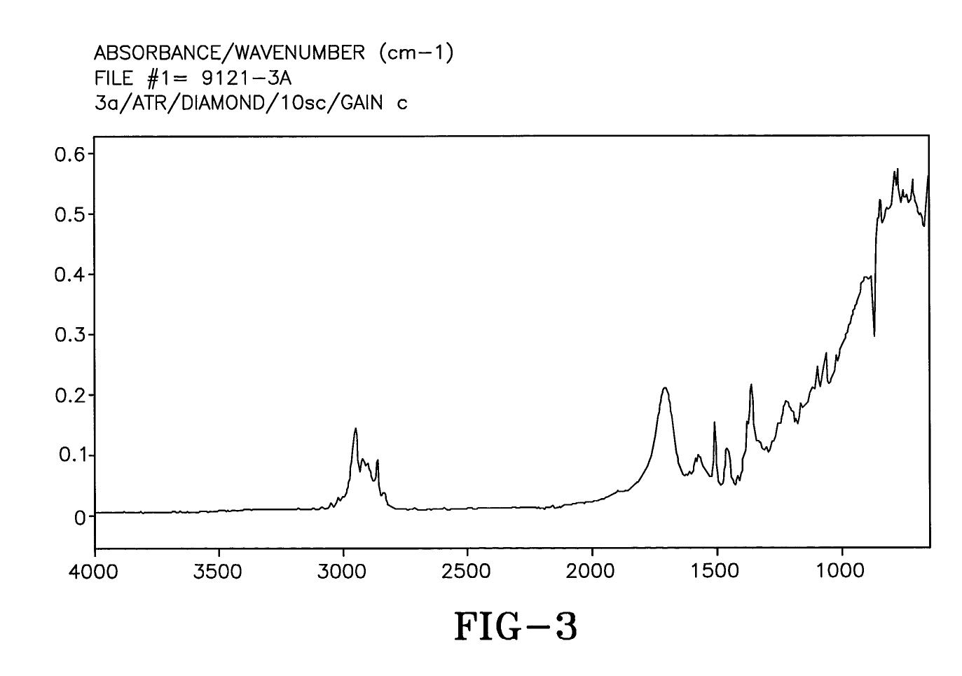

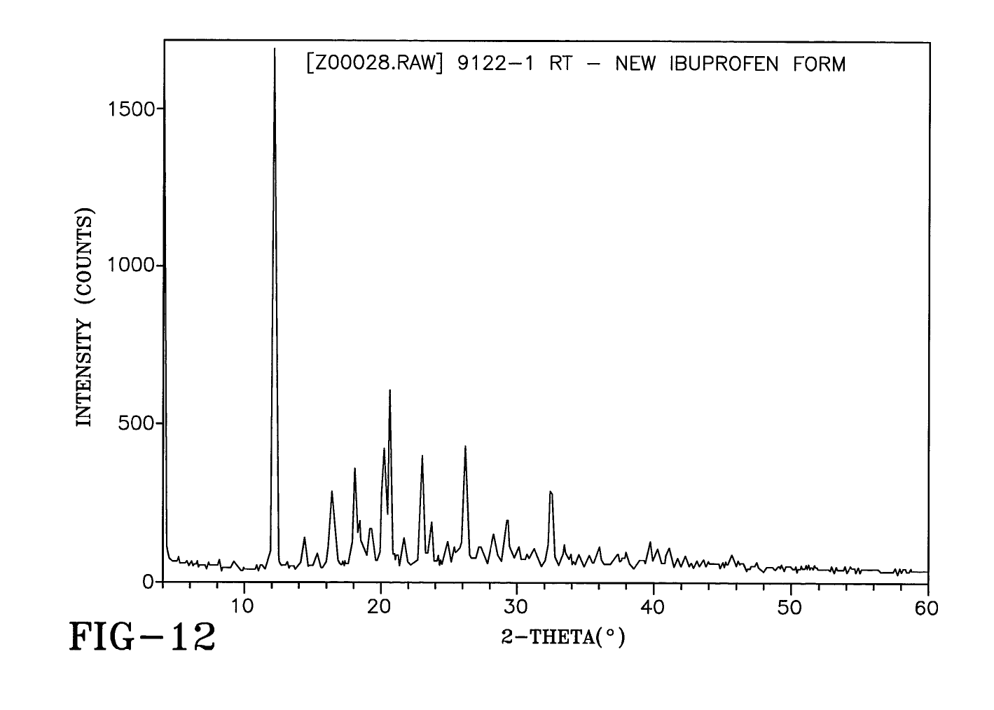

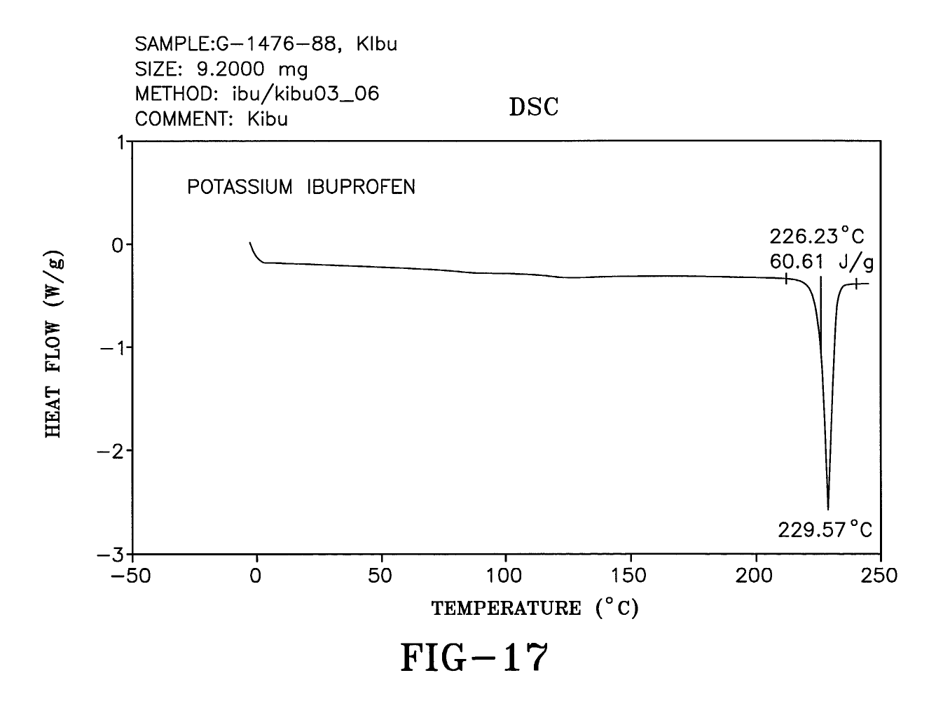

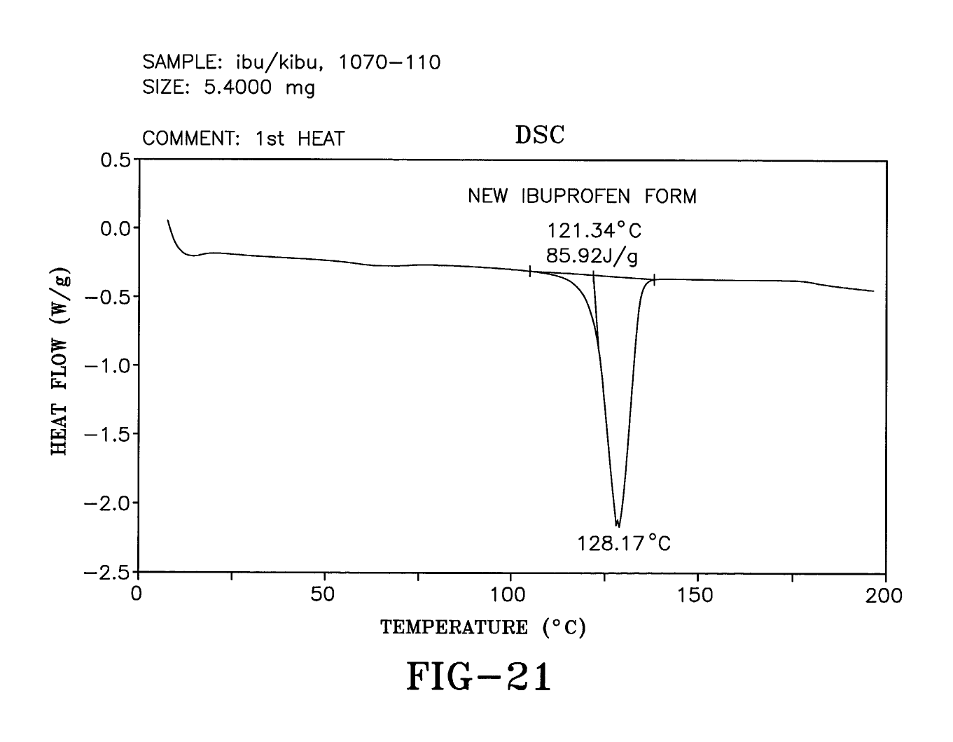

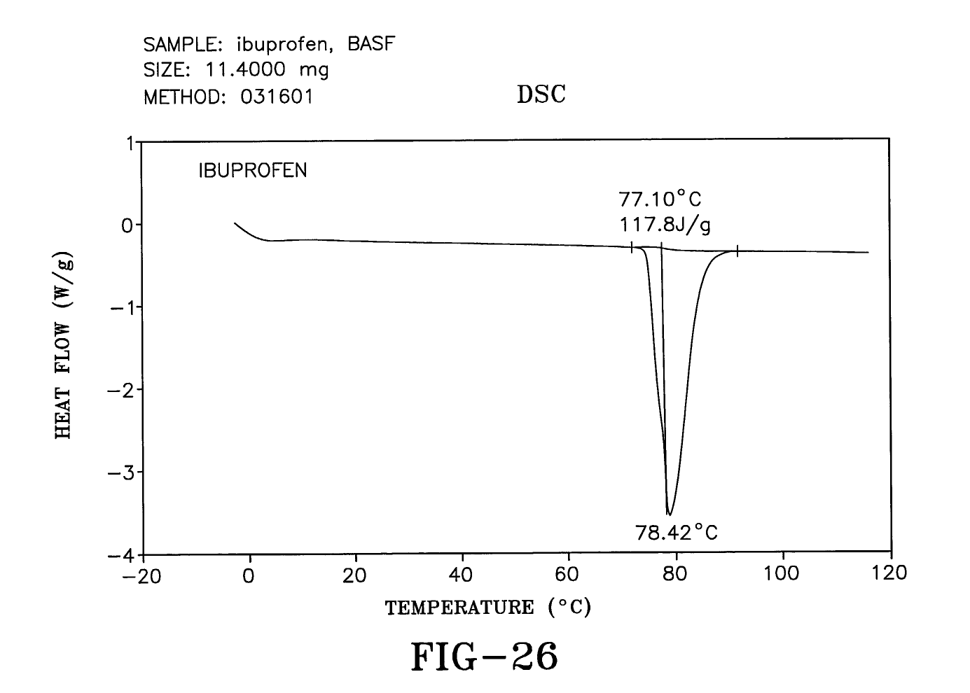

A particularly important embodiment of the present invention is a new ibuprofen form made out of a mixture of ibuprofen and any of its conjugate salts. When the cation of the conjugate salt is potassium, the new ibuprofen form exhibits: 1) an IR peak at 1706 cm⁻¹ shifted and broadened in absorbance relative to racemic ibuprofen free acid, 2) a characteristic absorbance profile from 1000 to 650 cm⁻¹ (FIG. 3) and 3) D-spacings of 21.1, 7.1, and 3.4 Å by X-ray diffraction 39 Å (corresponding 2 theta values of 4.2, 12.4 and 26.2, see FIG. 12). This ibuprofen form has a melting point range of 118° to 124° C. at a purity of at least 98% using differential scanning calorimetry (see FIG. 21). Ibuprofen, by comparison, has a melting point range of 74° C.-77° C. (see FIG. 26). And potassium ibuprofen has a melting point range of 224° C. to 228° C. (see FIG. 17). Melting points were visually confirmed by hot stage microscopy.

This ibuprofen form can be made in accordance with any of the procedures described above, and as set forth below in the Examples. The inventive ibuprofen can also be made by reacting a portion of ibuprofen with a base capable of reacting with such, for example, potassium hydroxide, in an aqueous medium followed by drying, for example, in a vacuum oven. This ibuprofen form, as set forth in the Examples, can also be made by melting the ibuprofen and reacting such with a base capable of reacting with such, for example, potassium hydroxide.

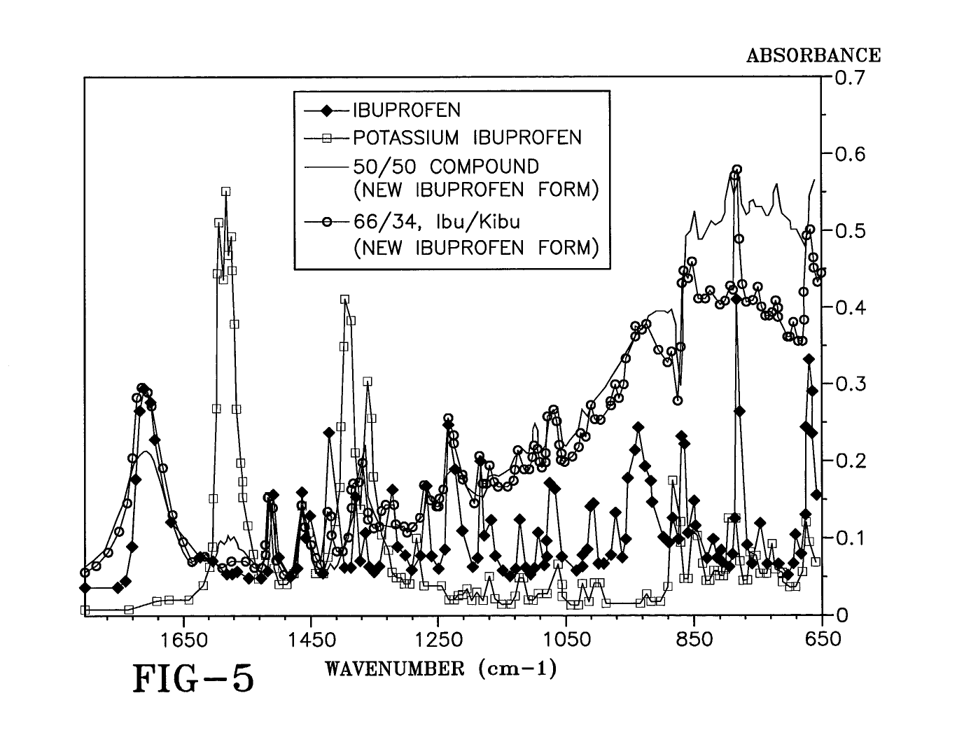

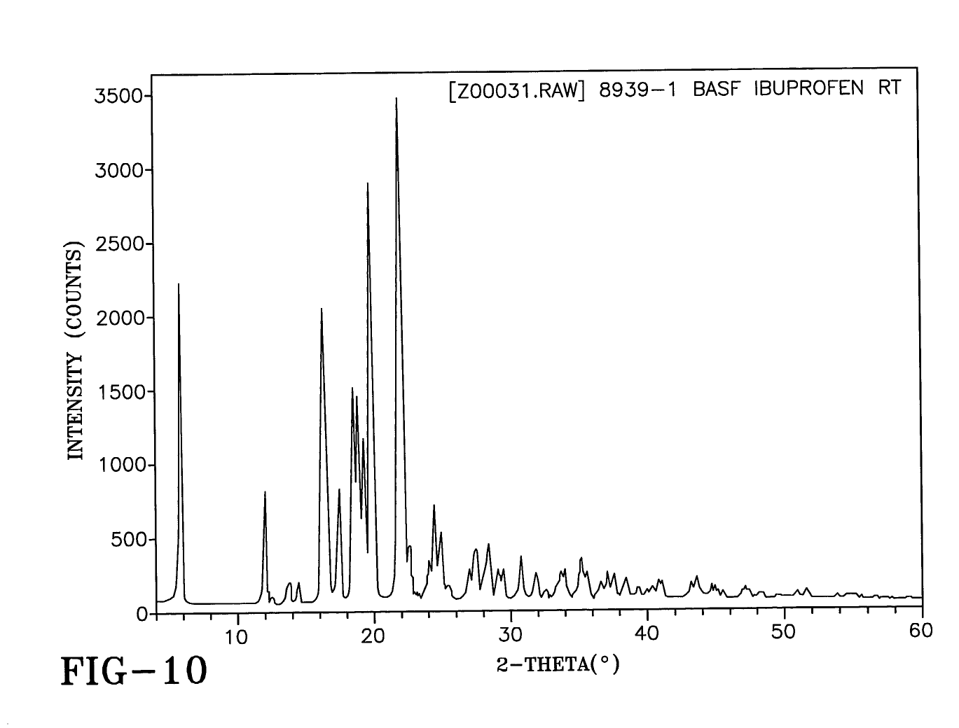

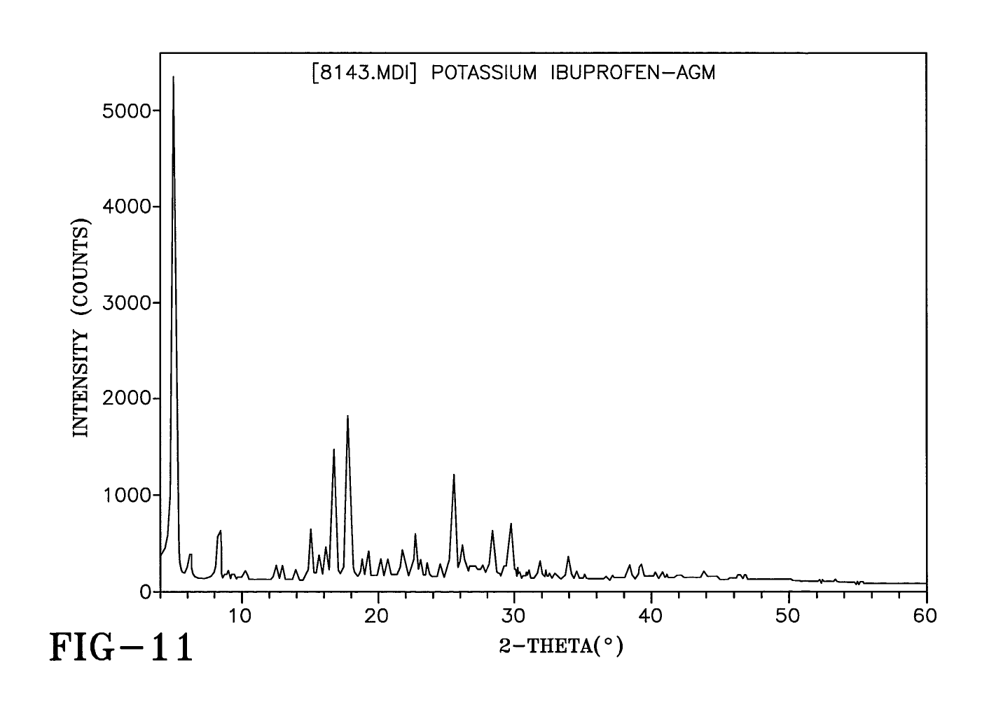

The IR spectra and X-ray diffraction pattern of the new ibuprofen form show clear differences when compared with the corresponding spectra for ibuprofen and potassium ibuprofen. For example, as shown in the IR overlay of FIG. 5, the IR spectral profile from 1000 to 650 cm⁻¹ for the new ibuprofen form of the invention having a potassium cation shows much stronger absorbance in that region with a sharp discontinuity at 871 cm⁻¹ which is very different from either ibuprofen or potassium ibuprofen. In addition, the major X-ray diffraction peaks for ibuprofen (FIG. 10) at 2 theta value of 6.0 and 16.5 do not appear in the X-ray diffraction pattern of the new ibuprofen form. Similarly, the major X-ray diffraction peaks for the new ibuprofen form (FIG. 12) at the 2 theta values of 4.2, 12.4 and 26.2 do not exist in the X-ray diffraction pattern of either ibuprofen or potassium ibuprofen (FIG. 11).

The new ibuprofen form of the present invention is a white powder in dried form. The new form of the pharmaceutically active agents, for example, the new ibuprofen form, can themselves be formulated into solid dosage forms such as, for example, tablets, capsules, suppositories, sprinkles or powders using procedures well known in the art. The new ibuprofen forms can be combined with other pharmaceutically acceptable excipients, as well as other medicaments. For example, solid dosage forms containing the new ibuprofen form can be prepared with antihistamines, decongestants, diuretics, antacids, prostagandins, analgesics, stimulants, expectorants, anesthetics and combinations thereof. Suitable antihistamines include terfenadine, astemizole, diphenhydramine, chlorpheniramine maleate, brompheniramine maleate, triprolidine, loratadine, desloratadine and the like. Examples of suitable decongestants include phenylpropanolamine, phenylephrine hydrochloride, pseudoephedrine hydrochloride and the like. Examples of suitable analgesics include acetaminophen, codeine, propoxyphene, hydrocodone and the like.

Suitable pharmaceutically acceptable excipients include granulating agents, binders, lubricants, colorants, fillers, flow aids, solvents, waxes, etc. Examples of such include calcium phosphate, starch, croscarmellose, polyvinylpyrrolidone, crospovidone, carboxymethylcellulose, methylcellulose, ethylcellulose, hydroxypropylmethylcellulose, hydroxypropylcellulose, lactose, maltodextrin, stearic acid, colloidal silica, magnesium sulfate, magnesium stearate, cellulose, hydrolyzed cellulose and microcrystalline cellulose.

The new forms of the present invention, particularly the new ibuprofen form, can also be formulated into a variety of other non-solid dosage forms such as, for example, liquid gel forms, elixirs, suspensions, solutions, injectables, inhalants, transdermal patches, topical creams and the like.

The new ibuprofen form of the present invention dissociates into ibuprofen in its free acid form. Importantly, the new ibuprofen form readily dissolves into aqueous medium as compared to ibuprofen. Thus, the new ibuprofen form of this invention provides an improved drug delivery system for ibuprofen and provides a method for improving the bioavailability of ibuprofen by providing a dosage form containing the new ibuprofen form of the invention and administering such to a patent whereby the new ibuprofen form dissolves into ibuprofen in vivo. The new ibuprofen form readily dissolves in water. It has been observed that placing the dried new form of ibuprofen in deionized water results in an apparent effervescence and a liquid is released as the new form dissolves. Under certain conditions of pH and concentration such that the solubility of the released ibuprofen is exceeded, at a distance from the surface of the particle, the liquid that was released was observed to further form fine feathered crystals of ibuprofen. It is believed when the new ibuprofen form dissolves in water and releases the counter ion, the pH of the local environment adjacent the particle is increased due to the dissolution of the counter ion, particularly, the potassium counter ion. In other words, the pH of the local environment adjacent the particle is increased due to the buffering capacity of the charged ibuprofen in the new form.

Bioavailability is defined as the rate and the extent of absorption of a bioactive agent into the blood for the distribution to its site of action. The low absorption rate of poorly water-soluble pharmaceutical active agents from the gastrointestinal tract is generally attributed to the poor solubility of these substances and to their poor absorption from gastrointestinal fluids.

Bioavailability of a poorly water-soluble drug is known to be enhanced if the drug is not present in a crystalline but in an amorphous physical state. In general, amorphous forms of a substance exhibit a higher solubility and a faster dissolution than their crystal forms since the dissolution of amorphous substances does not include the heat of solution required to overcome the crystal lattice energy. It is known, for example, that the antibiotic agent novobiocin can only be absorbed from the intestines after administration of the amorphous substance which has a solubility ten times higher than the crystalline agent (Mullins J. D., Macek T. J., J. Am. Pharm. Assoc., Sci. Ed. 49 (1960) 245).

The term ‘stable crystal’ means any crystal that is in a thermodynamically stable crystalline state and the term ‘metastable crystal’ means any crystalline state which is in a thermodynamically unstable crystalline state. The term ‘crystalline state’ is used referring to any of stable crystal, metastable crystal and amorphous (noncrystalline) solid. The term ‘heterogenous crystal’ means a crystal not in a singular crystalline state.

An organic substance which is in the solid physical state at room temperature is generally called a solid. At temperatures below their melting point, crystalline solids form a crystal lattice which is characterized by a three-dimensional order of atoms or molecules. The term “supercooling” which is synonymously called “undercooling” means that a solid, crystalline substance is not present in a solid, crystalline state at temperatures below its bulk melting point, but in a melt- or liquid-like state which is characterized by a more random distribution of atoms or molecules such as in liquids.

A supercooled or undercooled melt thus corresponds to an amorphous state which represents an amorphous liquid. The modified physical state might alternatively represent an amorphous but solid state such as the vitreous or glassy state. Since these amorphous physical states are not ordered contrary to the crystalline state, dissolution of amorphous substances does not require overcoming the crystal lattice energy. Thus, the dissolution rates of amorphous forms are usually greater.

The present invention is now described in more detail by reference to the following examples, but it should be understood that the invention is not construed as being limited thereto. Unless otherwise indicated herein, all parts, percentages, ratios and the like are by weight.

### EXAMPLE 1

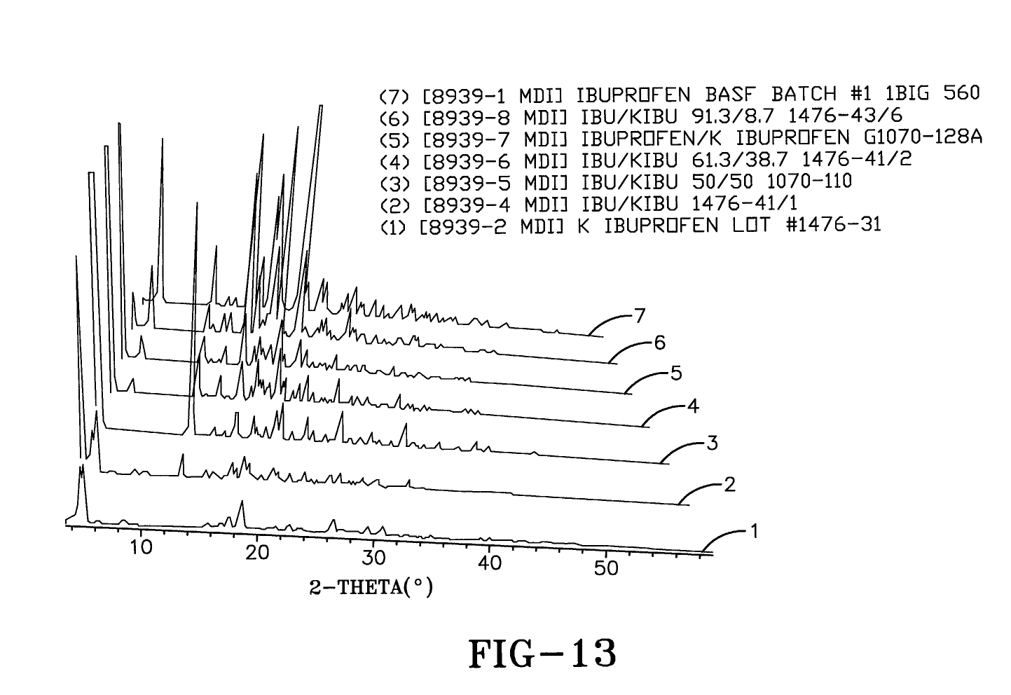

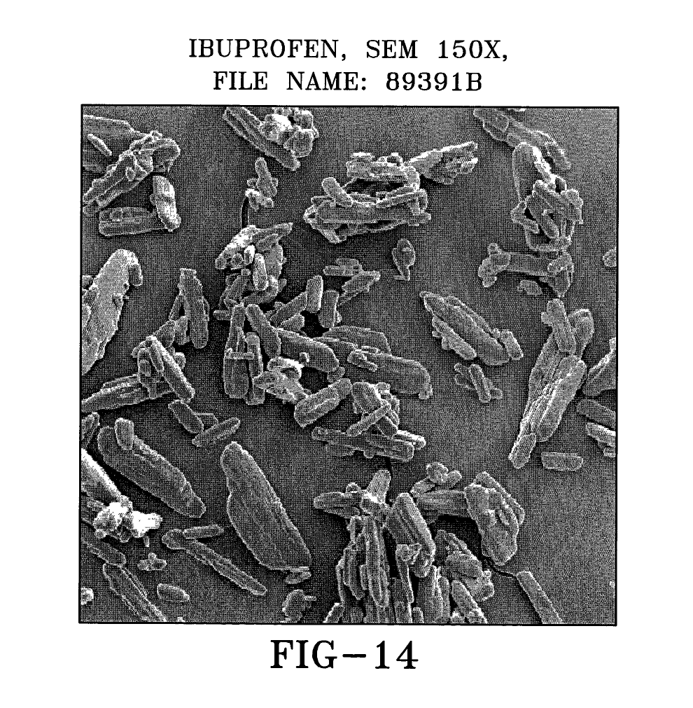

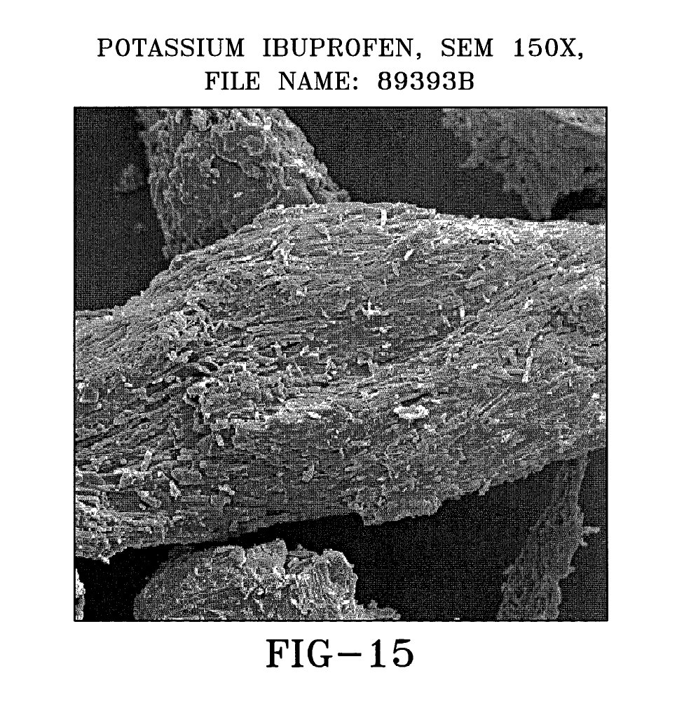

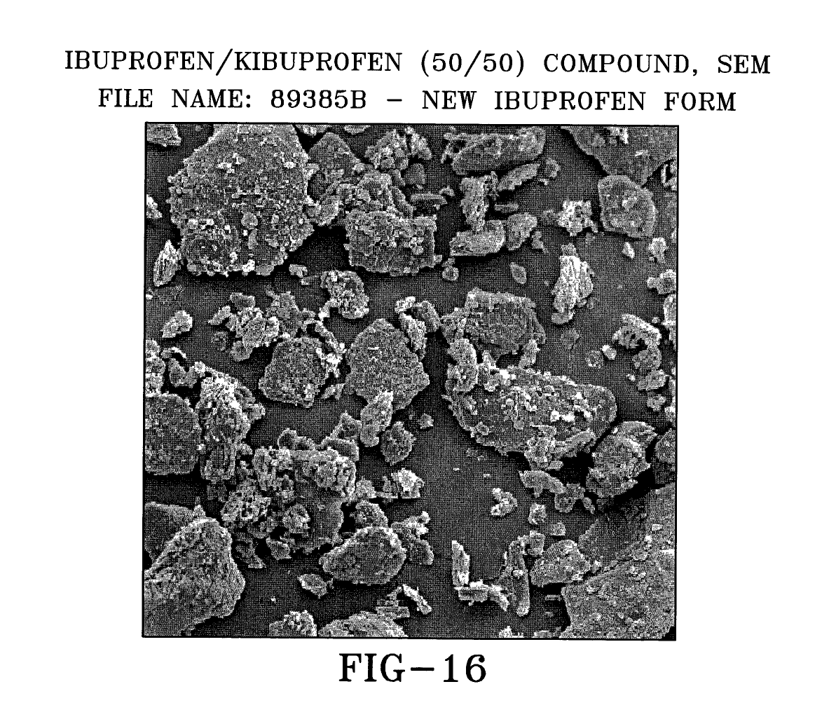

Potassium hydroxide pellets (110.42 grams @ 90% KOH, nominal 10% water, J. T. Baker) were added to deionized water (490.47 grams) in a stainless steel bowl while mixing using a Lightning mixer until the KOH was dissolved. Ibuprofen 38 (666.60 grams @>99.8% purity, BASF) was then added slowly to the mixture with vigorous agitation. Viscosity was observed to increase during the addition. The viscous gel-like material was transferred using a spatula to a shallow aluminum tray and dried in a vacuum oven (30 mmHg, 50° C.) for 8 days. The resultant waxy white powder was hand sieved through a 30 mesh screen. The DSC thermalgram at a heating rate of 10° C./min had a peak with an onset temperature of 121.3° C. and an enthalpy of 85.9 Joules/grm as shown in FIG. 21. The IR spectra is shown in FIG. 5. The X-ray is shown in FIG. 13.

### EXAMPLE 2

A composition having a 2 to 1 mole ratio of ibuprofen to potassium was prepared as follows. Into a 250 ml beaker was weighed 4.76 grams of KOH pellets (assayed at 83.46% KOH), 29.21 grams of Ibuprofen 38 (>99.8% purity) and 37 grams of deionized water. The mixture was stirred on a hot plate while heating to 82° C. and held at temperature for about 25 minutes. The translucent solution was a hazy gel when cooled. The sample was poured while hot into a container and dried in a vacuum oven to yield a white, waxy powder. Drying temperature was increased from room temperature (without vacuum) to up to 75° C. (with vacuum) during the drying process.

Dried particles of the new ibuprofen form having potassium as a cation as prepared in this example were placed on a Reichert-Jung hot stage microscope. Particles were observed to be bi-refringent plate-like crystals. When heated at 4° C./min above 110° C., the onset of melting occurred at 121° C. and was complete at 130° C. The sample was allowed to cool and resolidification was completed at 90° C. The second melt point was 123° C.

A drop of 1.0 normal HC1 solution was placed on a slide adjacent to particles prepared according to this example and observed under the hot stage microscope. As the solution wets the plate-like crystals, the plates become jagged and the plates turn into rod-like crystals similar in appearance to ibuprofen. The melting point of these rod-like crystals was observed to be 65 to 750 C when heated at a rate of about 4° C./min. The DSC thermalgram of ibuprofen 38 at a 10° C./min heating rate had a peak with an onset temperature of 77.1° C. and an enthalpy of 117.8 Joules/gram as shown in FIG. 26.

Potassium ibuprofen was prepared by adding 30.64 grams of potassium hydroxide (83.46%) to 200 grams of water in a stainless steel beaker. The solution was heated to 80° C. with mixing. While mixing, 94.02 grams of ibuprofen 38 was added and stirring continued for 30 minutes. The mixture was placed in a shallow pan and dried in a vacuum oven to recover potassium ibuprofen as a powder. The DSC thermalgram for potassium ibuprofen at a 10° C./min heating rate had a peak with an onset temperature of 226° C. and an enthalpy of 60.6 Joules/gram as shown in FIG. 17. The melting point of potassium ibuprofen was measured by a hot stage microscope. The melting onset was at 225° C. and melting was complete at 230° C. The sample was allowed to cool and resolidification was complete at 210° C.

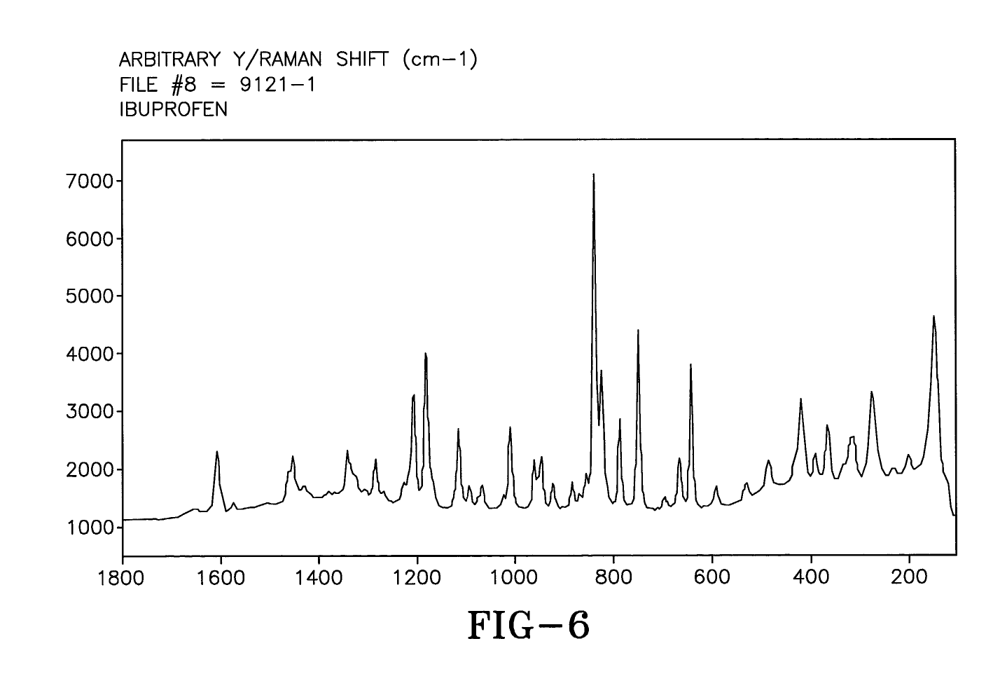

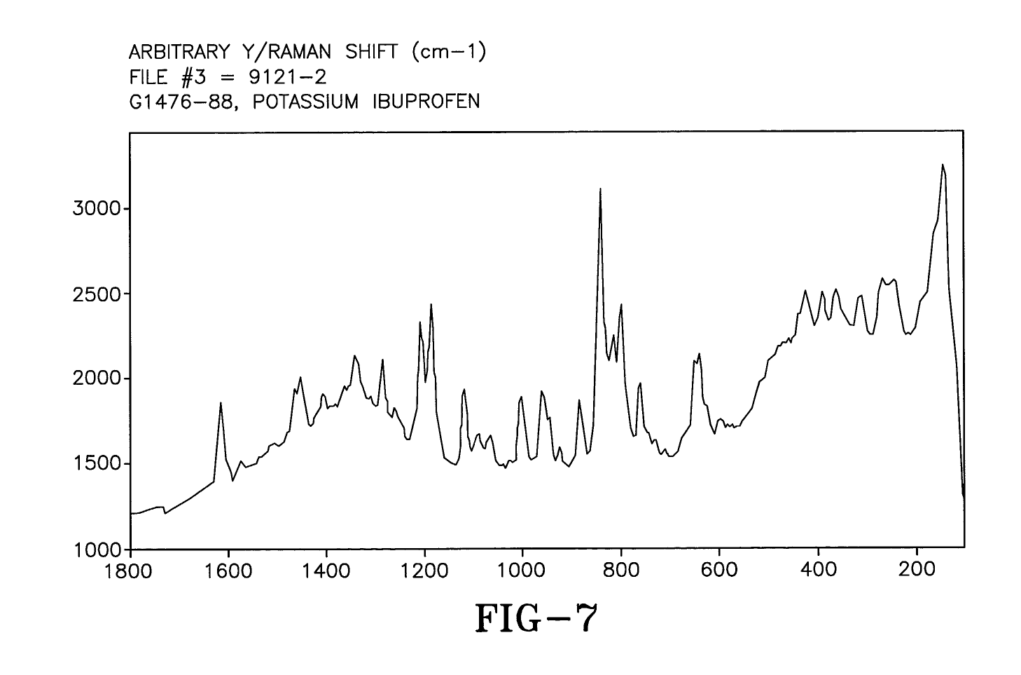

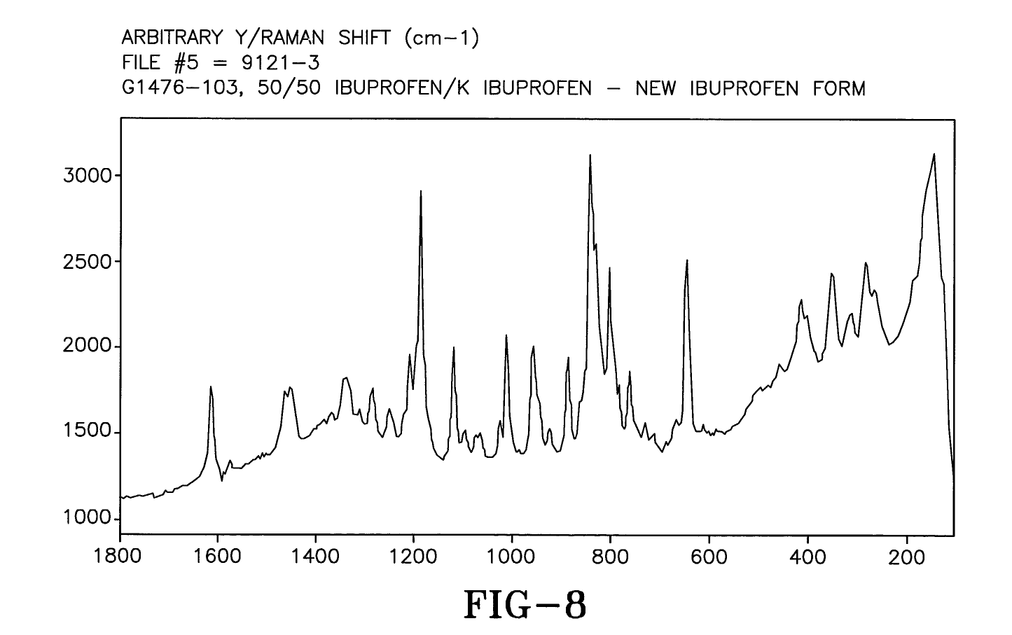

The X-ray diffraction patterns were obtained for the ibuprofen form having potassium as a cation, potassium ibuprofen and ibuprofen as shown in FIGS. 10-12. The x-ray data are tabulated in Tables 1-3. The major peaks for ibuprofen at 2 theta values of 6.0 and 22.2 and for potassium ibuprofen at 2 theta values of 5.3 and 8.5 do not appear in the x-ray diffraction pattern for the new ibuprofen form. Similarly, the major x-ray peaks for the new ibuprofen form at the 2 theta values of 4.2, 12.4 and 26.2 do not exist in the x-ray diffraction pattern for either ibuprofen or potassium ibuprofen. The Raman spectra as measured using a Kaiser optical system holoprobe (laser set at 785 nm and a charge couple detector) for the ibuprofen form having potassium as a cation in FIG. 8 is distinct from the Raman spectra for ibuprofen 38 and potassium ibuprofen as shown in FIGS. 6 and 7.

### EXAMPLE 3

A composition having a 2 to 1 mole ratio of ibuprofen to potassium was prepared as follows. Into a 500 ml stainless steel beaker was weighed 10.93 grams of KOH pellets, 107 grams of water and 67.07 grams of ibuprofen. The mixture was stirred on a hot plate at 300 rpms while heating to 90° C. The initial suspension gradually became a translucent light white solution. The sample was poured while hot to a container and dried in a vacuum oven to yield a white, waxy powder. Drying temperature was increased from room temperature (without vacuum) up to 50° C. (with vacuum) during the drying process.

### EXAMPLE 4

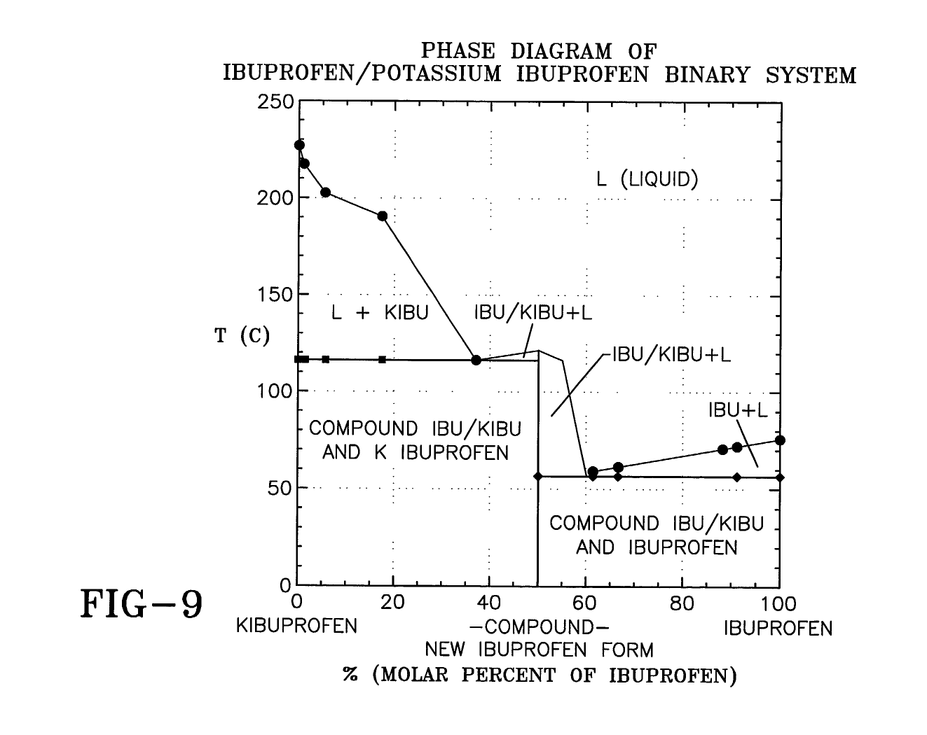

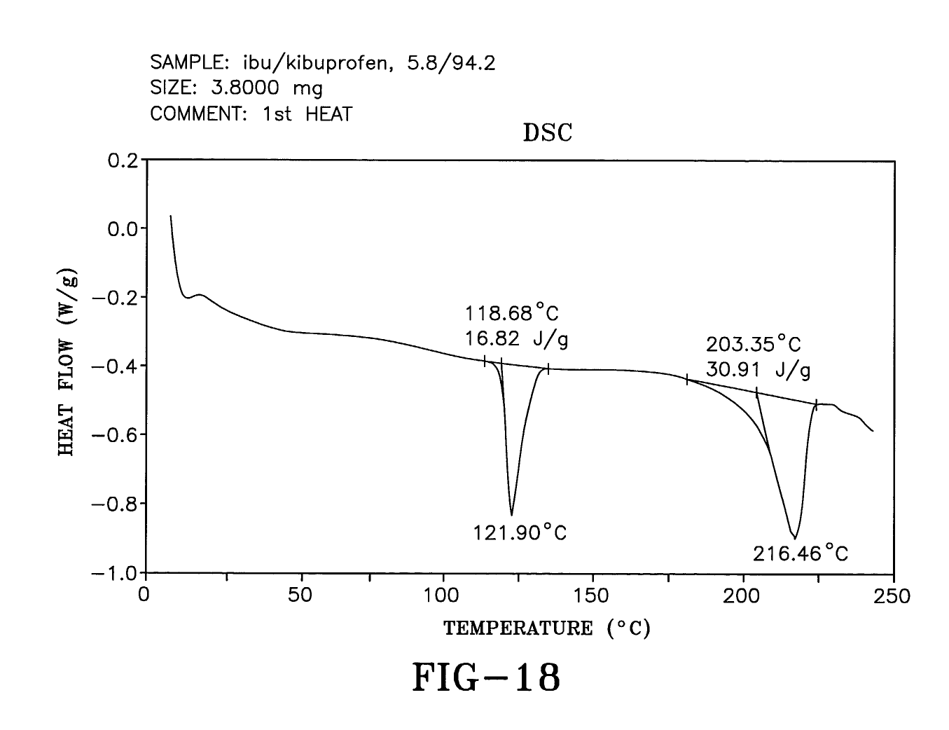

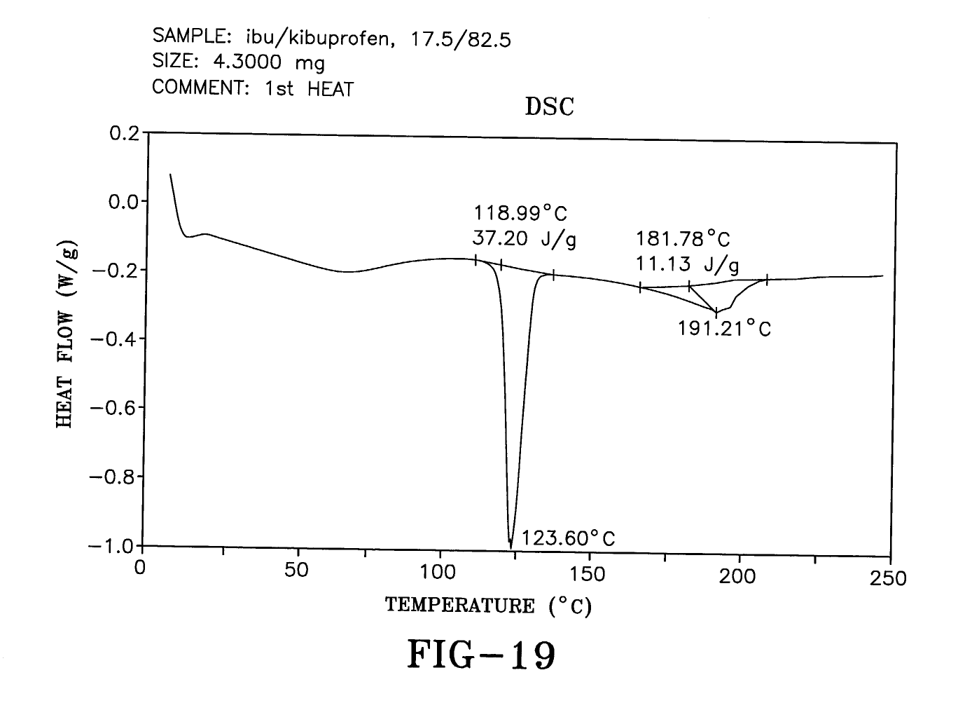

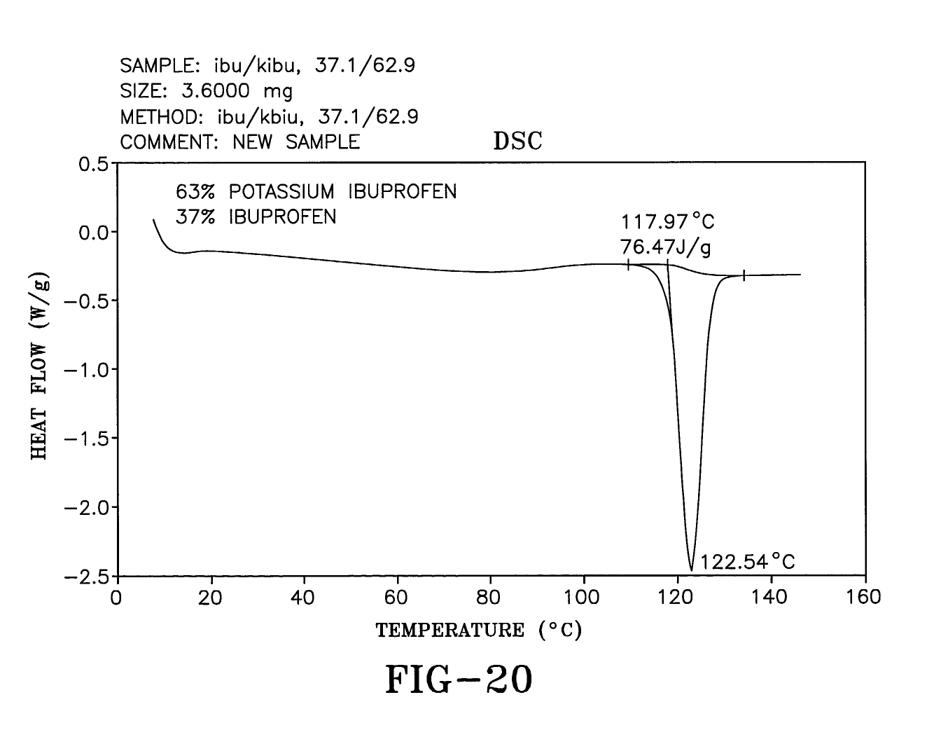

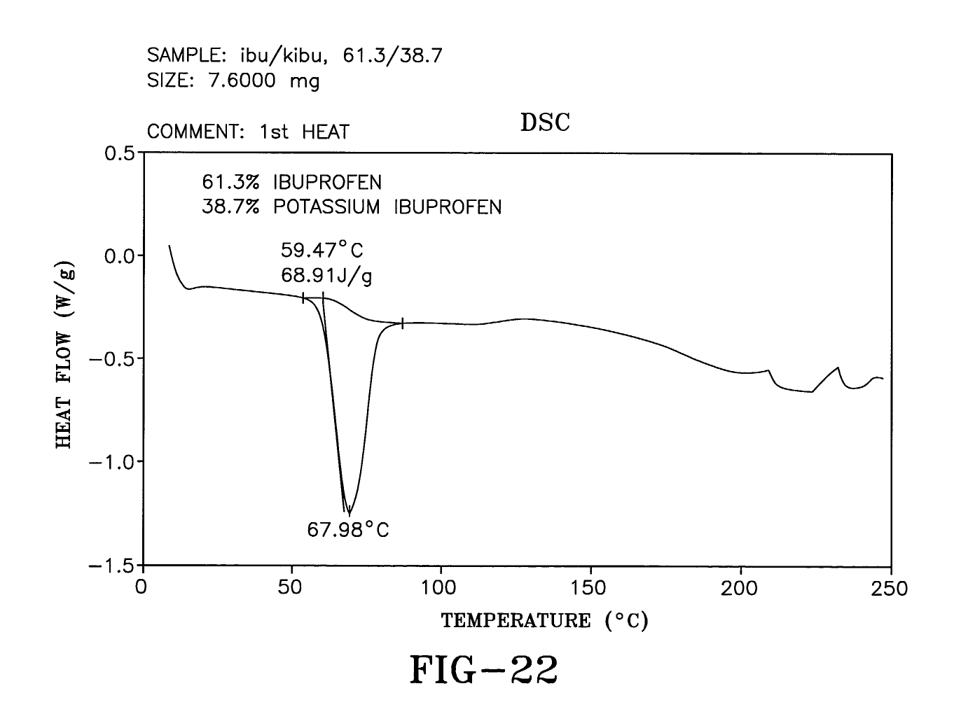

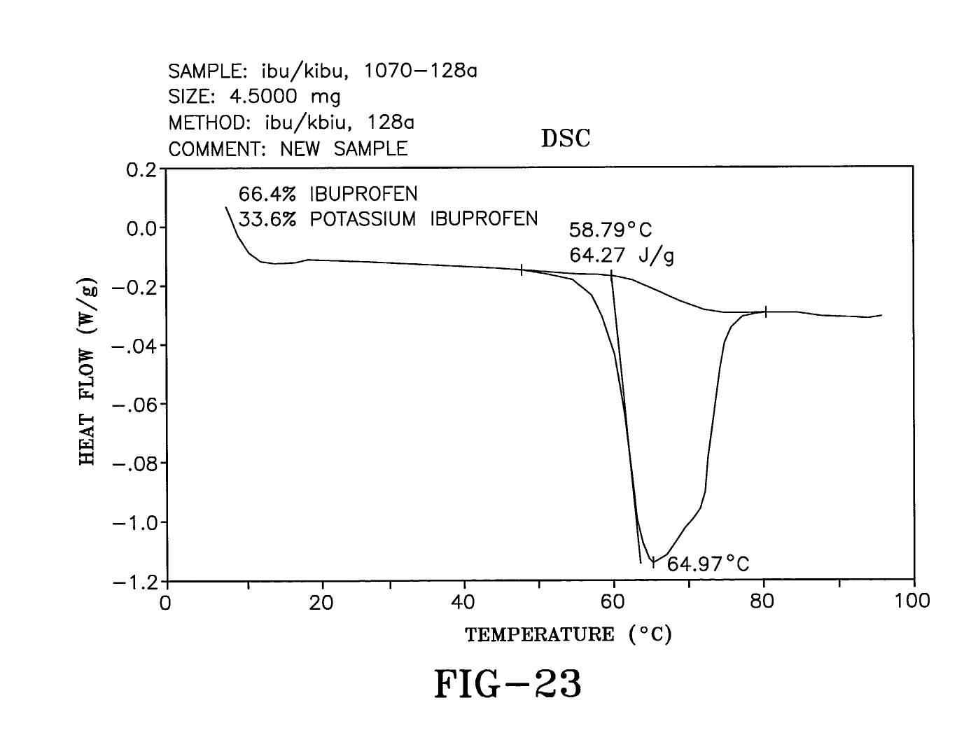

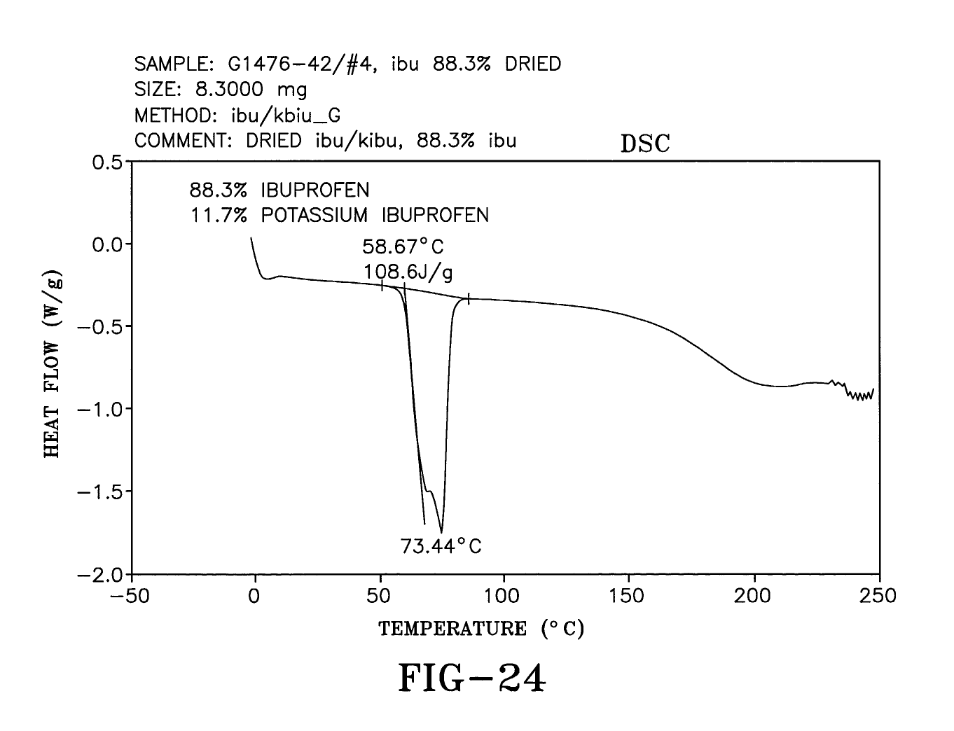

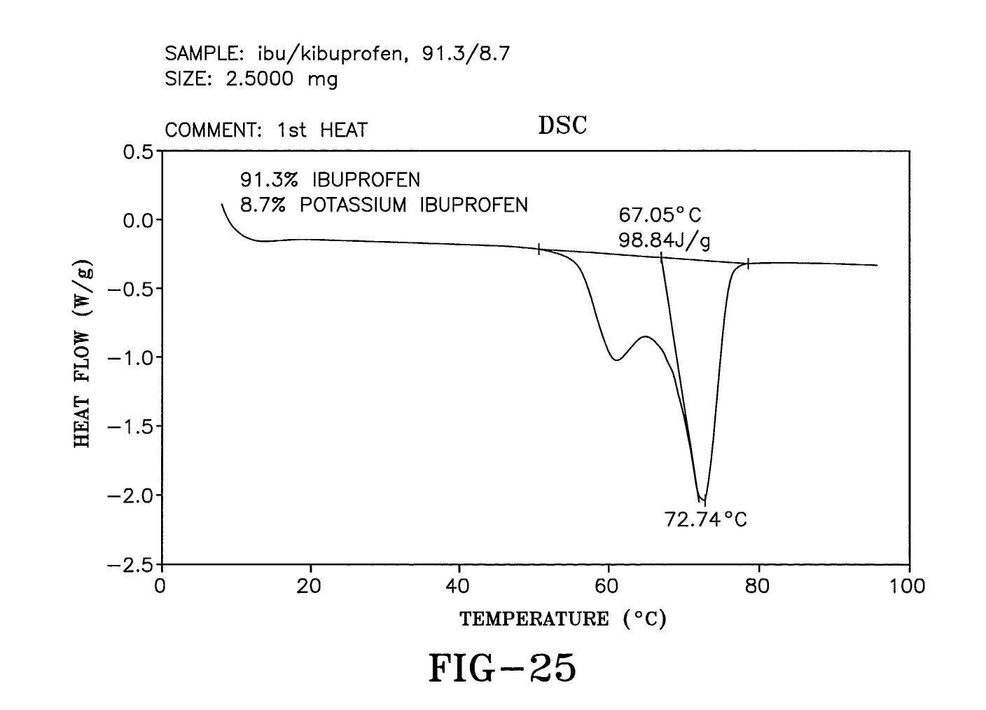

Dried powder compositions having varying mole ratios of ibuprofen to potassium were prepared according to the process of Example 3 by varying the relative molar amounts of ibuprofen 38 and potassium hydroxide. FIG. 5 is an overlay of FTIR curves for ibuprofen, potassium ibuprofen, the new ibuprofen form of the present invention (from Example 1 and 2) as well as the FTIR of the 66-34 molar mixture which shows the new ibuprofen form of the present invention wherein the cation is potassium. FIG. 13 is a 3-D overlay of X-ray diffraction patterns for ibuprofen (1), the new ibuprofen form of the invention wherein the cation is potassium (3) (Example 1), potassium ibuprofen (7) and mixtures thereof. FIGS. 17 to 20 and 22-25 show the DSC thermalgrams for these mixture compositions (17.5/82.5 IBU/KIBU; 37.1/62.9 IBU/KIBU; 61.3/38.7 IBU/KIBU; 66.4/33.6 IBU/KIBU; 88.3/11.7 IBU/KIBU; 91.3/8.7 IBU/KIBU; 94.2/5.8 KIBU/IBU). The results of the DSC data were used to construct a phase diagram (FIG. 9) for ibuprofen, potassium ibuprofen and the new ibuprofen form of the present invention wherein the cation is potassium showing the temperature and compositional ranges in which mixtures of the new ibuprofen form of the present invention wherein the cation is potassium are found with either potassium ibuprofen or ibuprofen respectively.

### EXAMPLE 5

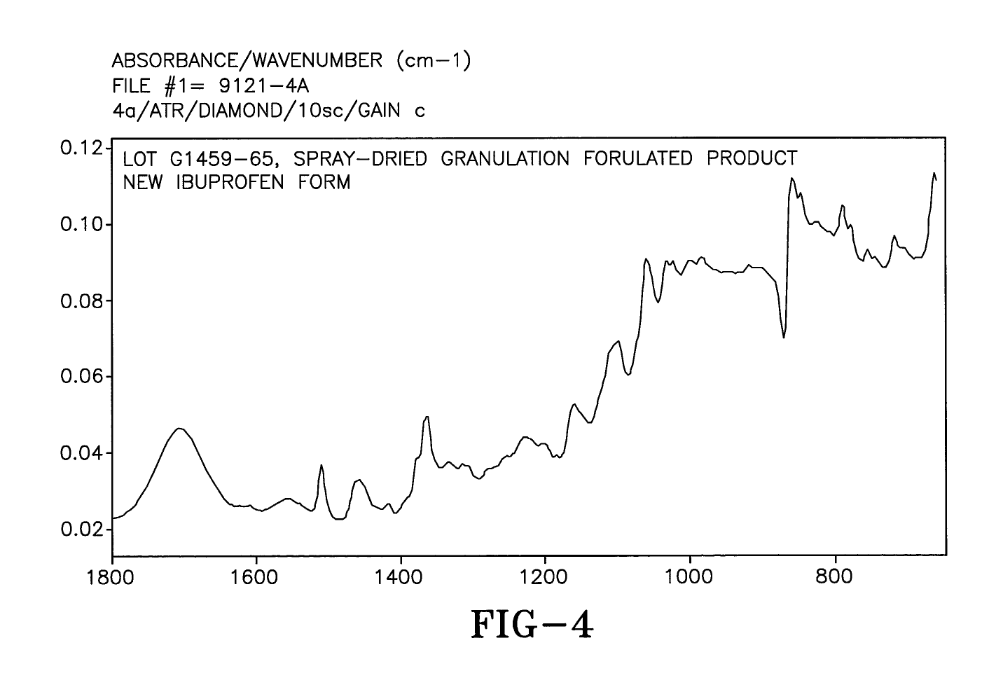

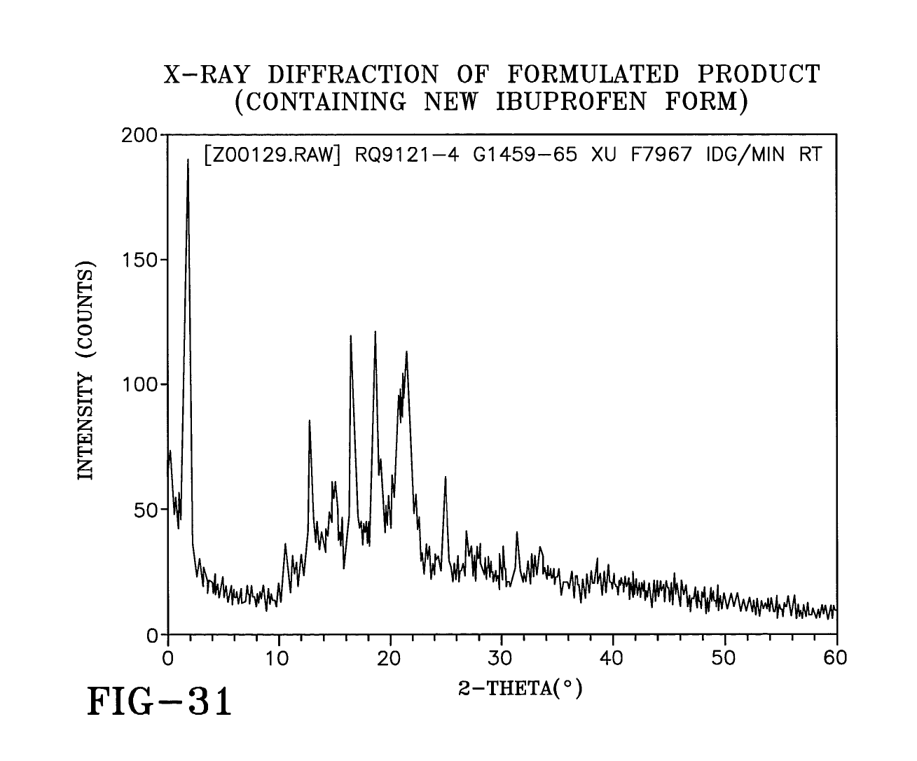

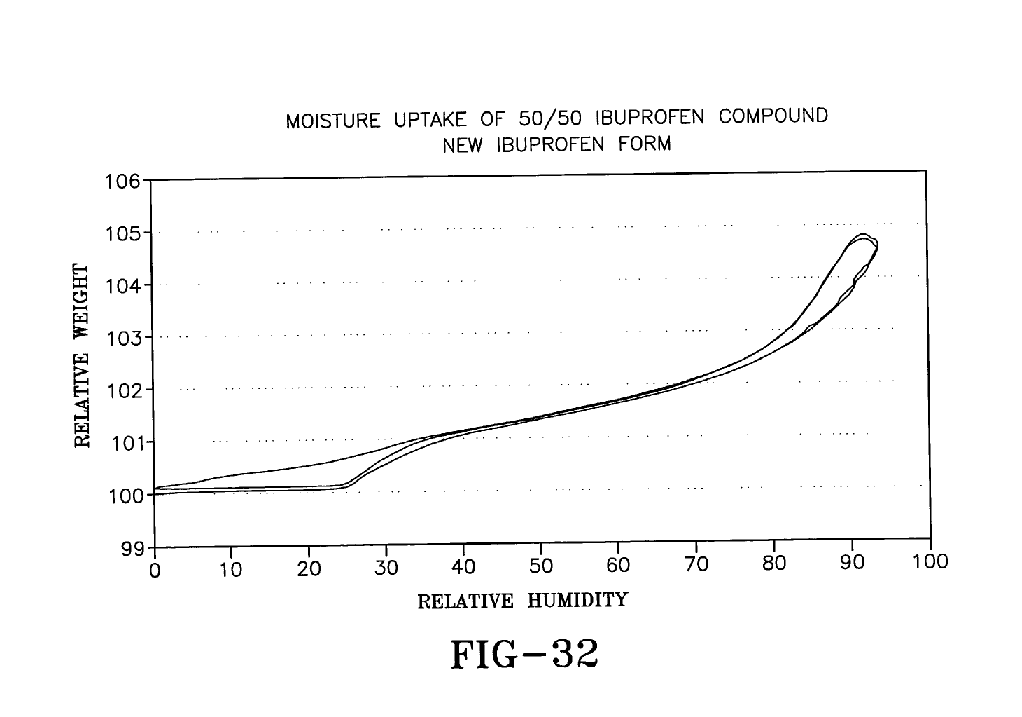

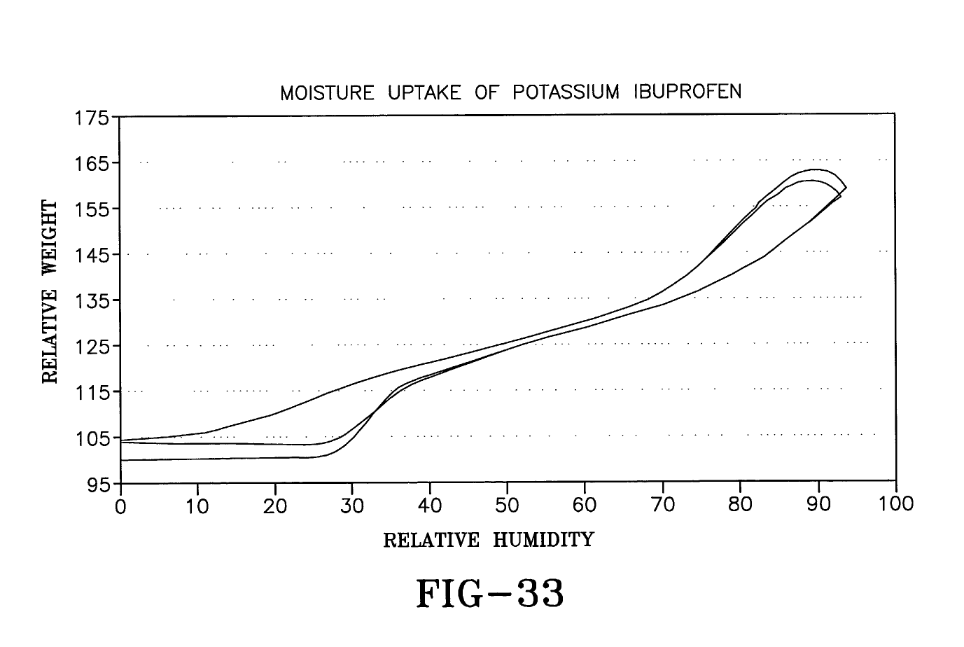

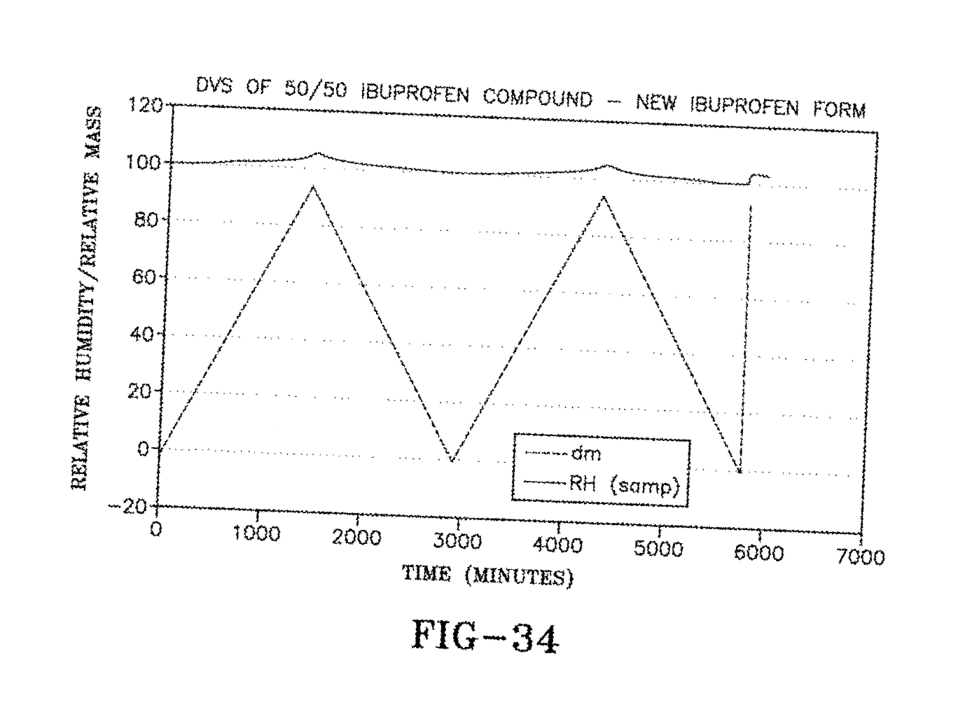

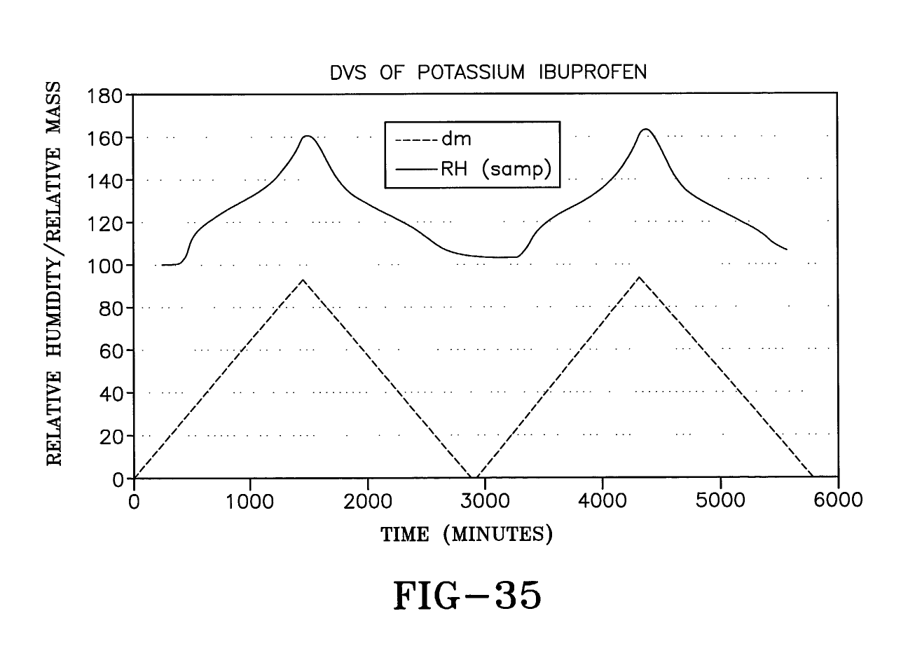

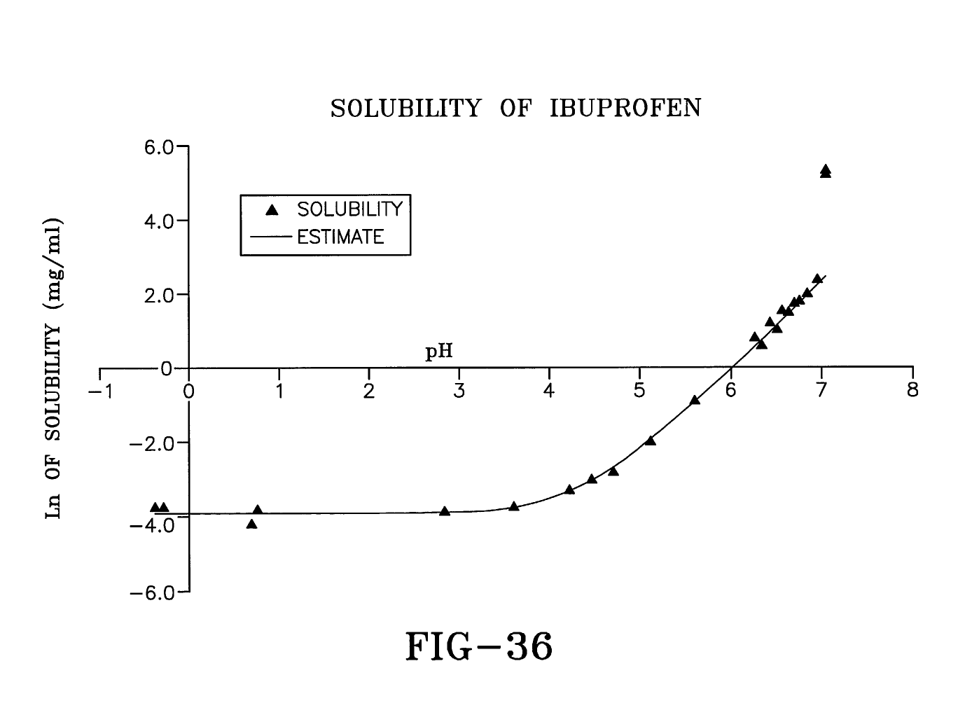

A slurry was prepared by adding through mixing, 25.275 kg of hydrolyzed cellulose wetcake (10.15 kg of solids) to deionized water (27.975 kg) followed by sequential addition of 0.375 kg of croscarmellose sodium (and stirring for 10 minutes), 0.25 kg of colloidal silicone dioxide (Cab-o-sil) M5P (and stirring for 10 minutes), 0.025 kg of sodium lauryl sulfate in deionized water (0.2 kg). A pre-mix of 12.5 kg of ibuprofen and 1.7 kg of potassium hydroxide (added as 1.94 kg of potassium hydroxide pellets) in 11.0 kg deionized water was then added to the slurry. The solids content of the slurry was 31.1% by weight. The mixture was spray-dried. The spray dryer had an inlet temperature of 210° F. and an outlet temperature of 115° F. The moisture content of the dried powder was 2.35 weight % at 49° C. The IR for the formulated product containing the new ibuprofen form is FIG. 4. The X-ray diffraction pattern for the formulated product containing the new ibuprofen form is FIG. 31 and the raw data is found in Table 4.

### EXAMPLE 6

Ibuprofen (50.1 grams) was melted on a hot plate and stirred at 100° C. Potassium hydroxide (8.1 grams) was dissolved in water (5.0 grams) and added to the molten ibuprofen with mixing. An additional two grams of water were added to flush the container and pipette. The mixture was cooled with stirring and nucleation was observed to begin at 64° C. The composition was transferred to a mortar and pestle and milled for several minutes. The composition had a melting point of 121° C. as a measured by heating on a hot stage microscope (heating rate of 4° C./min above 110° C.).

### EXAMPLE 7

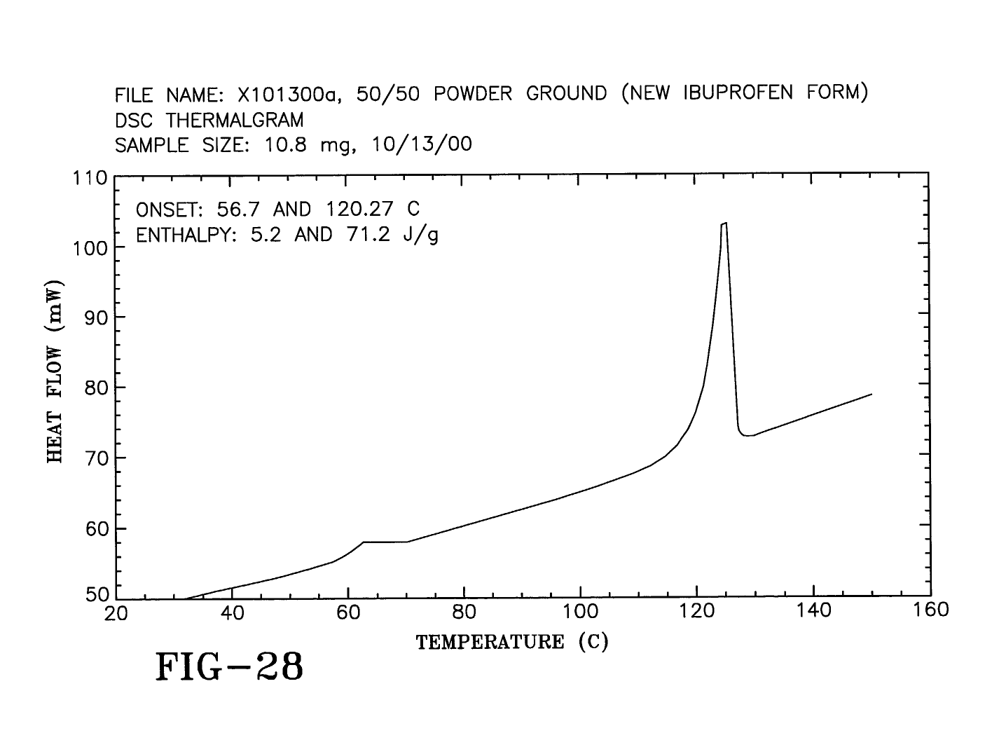

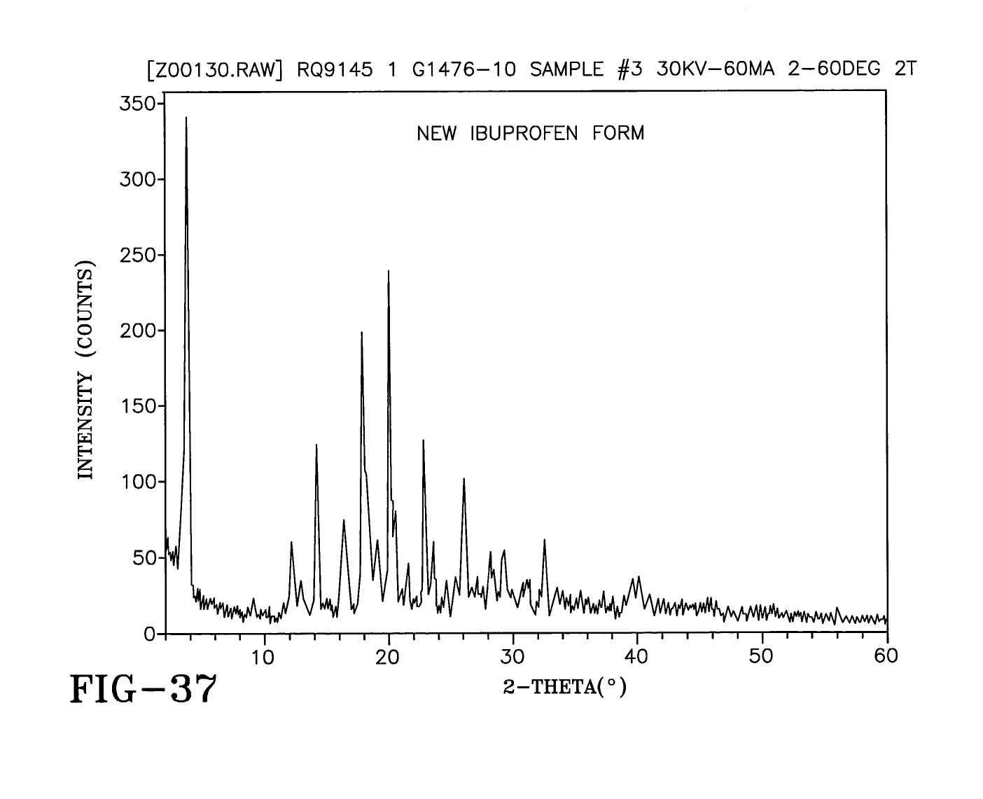

A mixture containing a 50-50 mole ratio of powdered ingredients was prepared by grinding together 516.8 grams of ibuprofen 38 and 610.6 grams of potassium ibuprofen using a mortar and pestle. The X-ray diffraction pattern of the mixture is shown in FIG. 37 and is tabulated in Table 5. A DSC thermalgram was obtained for a 10.8 mg sample of the ground powder heated at a rate of 10° C./minute from below 25° C. to 150° C. (FIG. 28). A small peak was observed with an onset temperature of 56° C. and an enthalpy of 5.2 Joules/gm as well as a larger peak with an onset temperature of 121.6° C. and an enthalpy of 78 Joules/gm corresponding to the new ibuprofen form of the present invention.

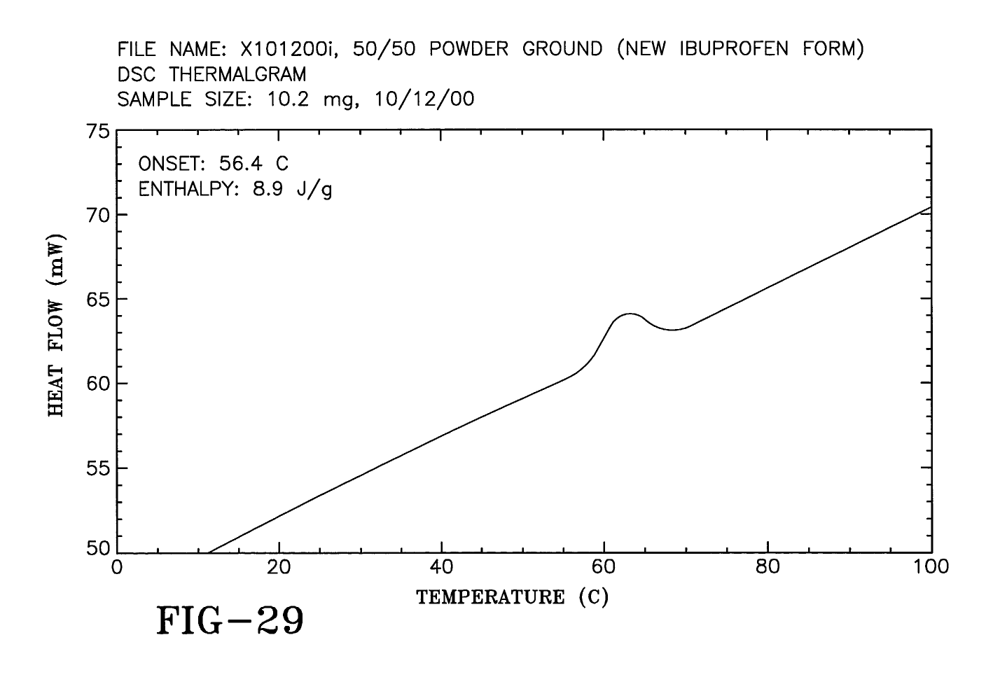

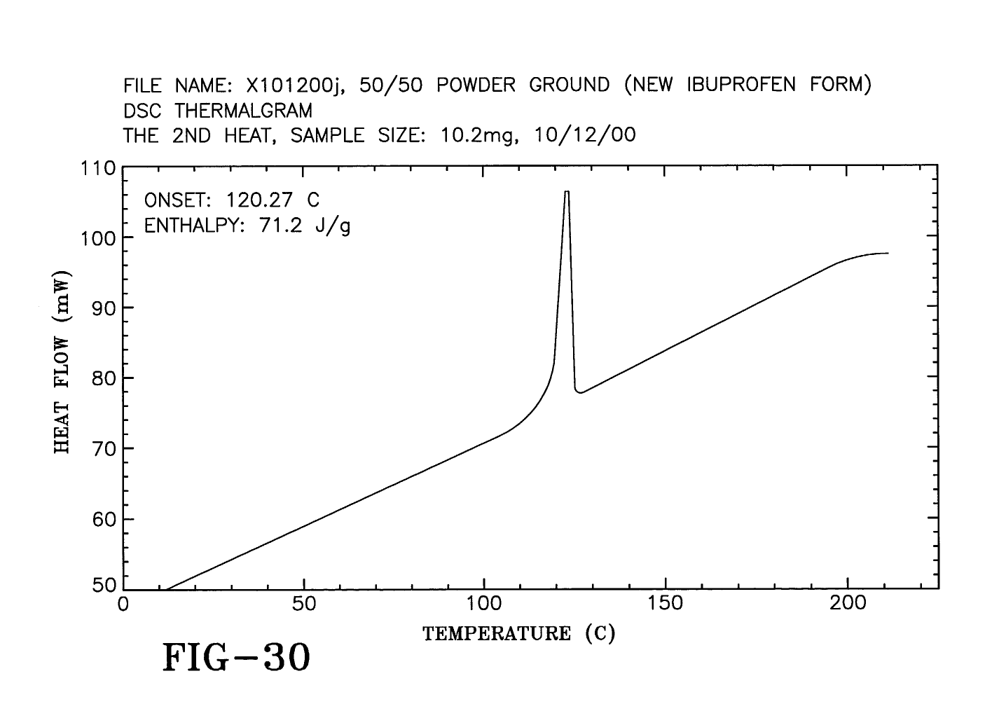

DSC thermalgrams were obtained using 10.2 mg samples of the mixture and the following heat treatment. The sample was first heated at 10° C./min to 100° C. then cooled at 10° C. (FIG. 29). A second heating cycle at a rate of 10° C./min from below 20° C. to 230° C. (FIG. 30). In the DSC thermalgram corresponding to the first heating as shown in FIG. 29, a peak was observed with an onset temperature of 56.4° C. and an enthalpy of 8.9 Joules/gram. The DSC thermalgram corresponding to the second heating, as shown in FIG. 30, no longer exhibited a peak at about 55° C., only a larger peak with an onset temperature of 120° C. and an enthalpy of 71 Joules/gram corresponding to the new ibuprofen form of this invention.

### EXAMPLE 8

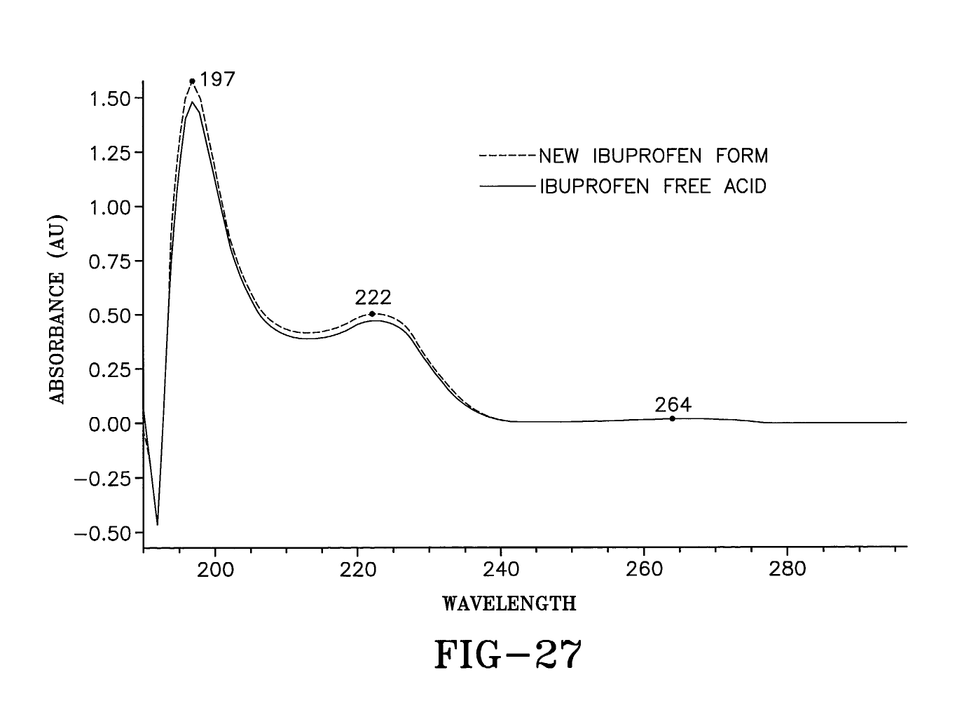

An ibuprofen standard was prepared by dissolving 111.8 grams of ibuprofen in 1000 ml of a solution of 50 millimolar sodium phosphate dibasic in water. A sample was prepared by dissolving 123.6 grams of the new ibuprofen form from Example 2 in 1000 ml of a solution of 50 millimolar sodium phosphate dibasic in water. UV absorbance was obtained for the ibuprofen standard and the sample as shown in FIG. 27. The absorbance at 222 nm was 0.50395 for the sample prepared using the new ibuprofen form and the absorbance was 0.47056 for ibuprofen standard. The slight offset in the absorbance observed at 222 nm was proportional to the calculated concentration difference in ibuprofen in the two solutions demonstrating that the new ibuprofen form dissolved to yield ibuprofen.

### EXAMPLE 9

The hygroscopic nature of potassium ibuprofen vs. the ibuprofen form obtained in Example 2 was characterized by the relative weight gain of the sample due to moisture pickup using a Dynamic Vapor Sorption Moisture Balance. Samples were cycled between 0% RH to 95% RH to 0% RH over 48 hours at 25° C. The potassium ibuprofen sample had a critical humidity of about 30%. At this humidity the sample dissolved and formed a gel. At 50% humidity, the relative weight gain due to moisture pickup was about 25% while at 70% humidity the relative weight gain was about 35%. The gel formation prevented recrystallization after the sample was dried. The ibuprofen form prepared in Example 2 had a critical humidity of about 30% on the first cycle. At 50% humidity the relative weight gain due to moisture pickup was about 1.4% and at 70% humidity the weight gain was bout 2%. No change in crystal appearance was observed. The new ibuprofen form having a potassium cation was significantly less hygroscopic compared to the potassium salt of ibuprofen.

Analytical Procedures

The following procedure was used for obtaining all melting point data provided herein. Differential scanning calorimetry (DSC), TA Instruments, DSC 2920, and Perkin-Elmer DSC System 7 were used to measure the melting points of ibuprofen, potassium ibuprofen, and ibuprofen-potassium ibuprofen mixture. The instrument was periodically calibrated for both heat and the temperature by using a certified standard, pure indium. The sample size was 4 to 13 mg; scan rate was 10° C./minute; pan was a nonhermetic aluminum pan; purge gas was ultra-pure grade nitrogen; and the temperature range investigated was 0 to 250° C. The melting point is defined as the point of intersection between the tangent of the onset slope of the melting peak and the baseline heat flow.

The IR spectrometer employed for scanning the samples was a BOMEM MB 102 equipped with a CsI beam splitter and DTGS detector operated at room temperature. Samples were scanned using an ASI Durasampler™ accessory equipped with an ATR 3-reflection diamond window. Spectra were acquired using the following parameters: 10 scans, spectral region 4000-660 cm⁻¹ and 4 cm⁻¹ resolution. For the IR analysis a portion of each sample was loaded onto the ATR window ‘as is’. To improve contact between sample particles and window, pressure is applied by screwing down the pressure applicator of the Durasampler.

The following procedure was used for obtaining the X-ray spectra described herein. X-ray diffraction (XRD), from a polycrystalline sample is described by the Bragg equation:

λ=2d sin θ

where λ is the wavelength of incident beam, d the spacing in angstroms between reflecting planes, and θ is the angle of reflection. The instrumentation for the analysis discussed here is a Rigaku powder X-ray Diffractometer employing Bragg-Brentano parafocusing geometry with a vertical goniometer in θ:2θ configuration. Instrument settings are as follows: generator power: 40 kV/30 mA; X-ray tube: Cu anode, long fine focusing; monochrometer: diffracted beam, graphite crystal; detector: sealed proportional; sample changer: both manual positioning and auto-positioning, without spinner, is employed; sample holders: front loaded cavity filled.

The sample preparation was as follows. Transferred about 0.6 grams of sample to a small agate mortar. Lightly ground to make a fine, uniform powder (30 seconds or less). Transferred ground powder to the cavity of a sample holder, and lightly packed in using a flat bladed spatula, or a glass slide. Mounted the holder on the goniometer, and close all panels on the safety enclosure. Using Rigaku PC based controller software the data was obtained using: scanning range: 2θ=2 degrees to 60 degrees (continuous); and scanning speed: 1 degree/minute. The spectra data was processed using Materials Data Incorporated (MDI) Jade software.

TABLE 1
Peak Search Report (35 Peaks, Max P/N = 28.6)
[Z00031.RAW] 8939-1 BASF IBUPROFEN RT

PEAK: 15-pts/Parabolic Filter, Threshold = 3.0, Cutoff = 0.1%,
BG = 3/1.0, Peak-Top = Summit

| 2-Theta | d(A) | BG | Height | 1% | Area | 1% | FWHM |
|---|---|---|---|---|---|---|---|
| 6.025 | 14.6575 | 64 | 2157 | 63.9 | 9205 | 33.2 | 0.181 |
| 12.134 | 7.2877 | 41 | 758 | 22.5 | 3920 | 14.1 | 0.22 |
| 13.834 | 6.396 | 52 | 132 | 3.9 | 960 | 3.5 | 0.309 |
| 14.546 | 6.0846 | 63 | 117 | 3.5 | 537 | 1.9 | 0.195 |
| 16.503 | 5.3671 | 56 | 1986 | 58.9 | 18873 | 68 | 0.404 |
| 17.546 | 5.0503 | 87 | 730 | 21.6 | 5095 | 18.4 | 0.297 |
| 18.695 | 4.7424 | 87 | 1412 | 41.9 | 11555 | 41.6 | 0.348 |
| 18.993 | 4.6688 | 93 | 1344 | 39.8 | 10400 | 37.5 | 0.329 |
| 19.348 | 4.5838 | 120 | 1030 | 30.5 | 7230 | 26 | 0.281 |
| 20.041 | 4.4268 | 83 | 2819 | 83.6 | 18711 | 67.4 | 0.282 |
| 22.209 | 3.9994 | 105 | 3373 | 100 | 27760 | 100 | 0.35 |
| 22.653 | 3.9221 | 92 | 335 | 9.9 | 2116 | 7.6 | 0.268 |
| 24.102 | 3.6894 | 92 | 229 | 6.8 | 3584 | 12.9 | 0.665 |
| 24.502 | 3.6301 | 133 | 567 | 16.8 | 5733 | 20.7 | 0.43 |
| 24.946 | 3.5664 | 69 | 443 | 13.1 | 4791 | 17.3 | 0.46 |
| 25.403 | 3.5033 | 69 | 89 | 2.6 | 654 | 2.4 | 0.312 |
| 26.998 | 3.2999 | 69 | 189 | 5.6 | 2083 | 7.5 | 0.468 |
| 27.452 | 3.2463 | 83 | 314 | 9.3 | 3885 | 14 | 0.526 |
| 28.442 | 3.1355 | 161 | 277 | 8.2 | 1694 | 6.1 | 0.26 |
| 29.453 | 3.0302 | 73 | 185 | 5.5 | 2277 | 8.2 | 0.523 |
| 30.801 | 2.9005 | 82 | 262 | 7.8 | 1776 | 6.4 | 0.288 |
| 31.866 | 2.806 | 85 | 149 | 4.4 | 1125 | 4.1 | 0.321 |
| 32.631 | 2.7419 | 84 | 35 | 1 | 138 | 0.5 | 0.168 |
| 33.999 | 2.6347 | 84 | 172 | 5.1 | 2202 | 7.9 | 0.544 |
| 35.255 | 2.5436 | 84 | 251 | 7.4 | 2992 | 10.8 | 0.507 |
| 36.65 | 2.4499 | 125 | 46 | 1.4 | 149 | 0.5 | 0.138 |
| 37.156 | 2.4178 | 89 | 152 | 4.5 | 2149 | 7.7 | 0.601 |
| 37.645 | 2.3874 | 113 | 113 | 3.4 | 627 | 2.3 | 0.236 |
| 38.445 | 2.3396 | 92 | 103 | 3.1 | 606 | 2.2 | 0.25 |
| 39.362 | 2.2872 | 90 | 47 | 1.4 | 200 | 0.7 | 0.181 |
| 40.858 | 2.2068 | 78 | 100 | 3 | 1342 | 4.8 | 0.57 |
| 43.606 | 2.0739 | 84 | 111 | 3.3 | 1276 | 4.6 | 0.489 |
| 44.757 | 2.0232 | 84 | 59 | 1.7 | 647 | 2.3 | 0.466 |
| 47.197 | 1.9241 | 62 | 62 | 1.8 | 678 | 2.4 | 0.465 |
| 51.665 | 1.7677 | 60 | 48 | 1.4 | 414 | 1.5 | 0.367 |

TABLE 2
Peak Search Report (29 Peaks, Max P/N = 14.7)
[Z00042.RAW] 9121-2 XU G1476-88 Potassium Ibuprofen

PEAK: 13-pts/Parabolic Filter, Threshold = 3.0, Cutoff = 0.1%,
BG = 3/1.0, Peak-Top = Summit

| 2-Theta | d(A) | BG | Height | 1% | Area | 1% | FWHM |
|---|---|---|---|---|---|---|---|
| 5.333 | 16.5573 | 68 | 929 | 100 | 6484 | 100 | 0.297 |
| 6.35 | 13.9065 | 48 | 32 | 3.4 | 91 | 1.4 | 0.121 |
| 8.451 | 10.454 | 34 | 72 | 7.8 | 407 | 6.3 | 0.24 |
| 12.693 | 6.9683 | 26 | 14 | 1.5 | 135 | 2.1 | 0.386 |
| 14.062 | 6.2929 | 28 | 22 | 2.4 | 98 | 1.5 | 0.178 |
| 15.15 | 5.8433 | 40 | 56 | 6 | 336 | 5.2 | 0.255 |
| 16.258 | 5.4473 | 57 | 56 | 5.7 | 338 | 5.2 | 0.271 |
| 16.851 | 5.2571 | 63 | 132 | 14.2 | 935 | 14.4 | 0.301 |
| 17.851 | 4.9647 | 57 | 349 | 37.6 | 2626 | 40.5 | 0.32 |
| 18.897 | 4.6923 | 44 | 25 | 2.7 | 253 | 3.9 | 0.405 |
| 19.338 | 4.5861 | 44 | 33 | 3.6 | 254 | 3.9 | 0.327 |
| 20.199 | 4.3927 | 47 | 27 | 2.9 | 374 | 5.8 | 0.589 |
| 20.793 | 4.2685 | 49 | 34 | 3.7 | 382 | 5.9 | 0.478 |
| 21.806 | 4.0723 | 56 | 85 | 9.1 | 462 | 7.1 | 0.231 |
| 22.744 | 3.9065 | 56 | 47 | 5.1 | 336 | 5.2 | 0.304 |
| 24.575 | 3.6195 | 52 | 24 | 2.6 | 75 | 1.2 | 0.125 |
| 25.55 | 3.4835 | 65 | 150 | 16.1 | 1683 | 26 | 0.477 |
| 26.053 | 3.4173 | 76 | 33 | 3.6 | 237 | 3.7 | 0.287 |
| 28.446 | 3.1351 | 60 | 74 | 8 | 523 | 8.1 | 0.3 |
| 29.793 | 2.9964 | 51 | 89 | 9.6 | 953 | 14.7 | 0.455 |
| 32.099 | 2.7862 | 42 | 20 | 2.2 | 272 | 4.2 | 0.544 |
| 33.949 | 2.6385 | 39 | 31 | 3.3 | 178 | 2.7 | 0.244 |
| 38.405 | 2.3419 | 40 | 18 | 1.9 | 125 | 1.9 | 0.278 |
| 39.203 | 2.2961 | 46 | 10 | 1.1 | 40 | 0.6 | 0.16 |
| 41.999 | 2.1495 | 37 | 10 | 1.1 | 84 | 1.3 | 0.336 |
| 43.955 | 2.0582 | 37 | 16 | 1.7 | 158 | 2.4 | 0.395 |
| 46.427 | 1.9542 | 30 | 14 | 1.5 | 144 | 2.2 | 0.411 |
| 49.096 | 1.854 | 28 | 12 | 1.3 | 132 | 2 | 0.44 |
| 53.17 | 1.7212 | 22 | 9 | 1 | 65 | 1 | 0.289 |

TABLE 3
Peak Search Report (29 Peaks, Max P/N = 46.7
[Z00029.RAW] 9122-2 RT 50/50 compound, G 1476-103

PEAK: 15-pts/Parabolic Filter, Threshold = 3.0, Cutoff = 0.1%,
BG = 3/1.0, Peak-Top = Summit

| 2-Theta | d(A) | BG | Height | 1% | Area | 1% | FWHM |
|---|---|---|---|---|---|---|---|
| 4.183 | 21.1074 | 55 | 8796 | 100 | 39228 | 100 | 0.19 |
| 9.381 | 9.42 | 32 | 34 | 0.4 | 115 | 0.3 | 0.144 |
| 12.444 | 7.107 | 38 | 878 | 10 | 4543 | 11.6 | 0.22 |
| 14.597 | 6.0634 | 41 | 134 | 1.5 | 837 | 2.1 | 0.265 |
| 15.523 | 5.7038 | 39 | 28 | 0.3 | 154 | 0.4 | 0.234 |
| 16.6 | 5.3359 | 43 | 166 | 1.9 | 1346 | 3.4 | 0.345 |
| 18.2 | 4.8703 | 45 | 383 | 4.4 | 3506 | 8.9 | 0.389 |
| 19.35 | 4.5835 | 89 | 87 | 1 | 402 | 1 | 0.196 |
| 20.348 | 4.3608 | 73 | 448 | 5.1 | 3354 | 8.6 | 0.318 |
| 20.759 | 4.2754 | 76 | 356 | 4 | 2043 | 5.2 | 0.244 |
| 21.799 | 4.0738 | 60 | 110 | 1.3 | 516 | 1.3 | 0.199 |
| 23.099 | 3.8473 | 59 | 369 | 4.2 | 2433 | 6.2 | 0.28 |
| 23.753 | 3.7428 | 71 | 130 | 1.5 | 685 | 1.7 | 0.224 |
| 25.642 | 3.4712 | 75 | 33 | .04 | 349 | 0.9 | 0.449 |
| 26.251 | 3.3921 | 74 | 372 | 4.2 | 2613 | 6.7 | 0.299 |
| 27.309 | 3.263 | 72 | 42 | 0.5 | 229 | 0.6 | 0.232 |
| 28.296 | 3.1514 | 71 | 99 | 1.1 | 647 | 1.6 | 0.278 |
| 29.305 | 3.0451 | 86 | 131 | 1.5 | 638 | 1.6 | 0.207 |
| 31.22 | 2.8625 | 66 | 46 | 0.5 | 399 | 1 | 0.369 |
| 32.598 | 2.7447 | 62 | 190 | 2.2 | 1278 | 3.3 | 0.286 |
| 35.511 | 2.6719 | 64 | 43 | 0.5 | 353 | 0.9 | 0.349 |
| 35.403 | 2.5333 | 58 | 33 | 0.4 | 398 | 1 | 0.513 |
| 36.053 | 2.4891 | 56 | 40 | 0.5 | 496 | 1.3 | 0.527 |
| 37.355 | 2.4053 | 55 | 40 | 0.5 | 478 | 1.2 | 0.508 |
| 38.178 | 2.3554 | 57 | 30 | 0.3 | 147 | 0.4 | 0.208 |
| 39.797 | 2.2632 | 58 | 73 | 0.8 | 901 | 2.3 | 0.525 |
| 40.251 | 2.2387 | 65 | 44 | 0.5 | 361 | 0.9 | 0.349 |
| 41.184 | 2.1901 | 61 | 32 | 0.4 | 163 | 0.4 | 0.216 |
| 45.85 | 1.9775 | 50 | 37 | 0.4 | 439 | 1.1 | 0.504 |

TABLE 4
Peak Search Report (23Peaks, Max P/N = 6.0
RQ9121-4 G1459-65, X-ray data of formulated product

PEAK: 17-pts/Parabolic Filter, Threshold = 3.0, Cutoff = 0.1%,
BG = 3/1.0, Peak-Top = Summit

| 2-Theta | d(A) | BG | Height | 1% | Area | 1% | FWHM |
|---|---|---|---|---|---|---|---|
| 4.00 | 22.0 | 25 | 167 | 100 | 1586 | 100 | 0.404 |
| 12.154 | 7.2762 | 18 | 18 | 10.8 | 83 | 5.2 | 0.184 |
| 12.836 | 6.891 | 22 | 9 | 5.4 | 98 | 6.2 | 0.436 |
| 14.44 | 6.1289 | 33 | 53 | 31.7 | 403 | 25.4 | 0.323 |
| 14.963 | 5.9159 | 36 | 9 | 5.4 | 91 | 5.7 | 0.404 |
| 16.501 | 5.3678 | 34 | 27 | 16.2 | 334 | 21.1 | 0.526 |
| 18.045 | 4.91 19 | 36 | 84 | 50.3 | 818 | 51.6 | 0.414 |
| 20.101 | 4.4138 | 42 | 79 | 47.3 | 742 | 46.8 | 0.399 |
| 22.456 | 3.9559 | 57 | 48 | 28.7 | 929 | 58.6 | 0.823 |
| 22.807 | 3.8959 | 56 | 58 | 34.7 | 898 | 56.6 | 0.658 |
| 23.503 | 3.7821 | 27 | 29 | 17.4 | 222 | 14 | 0.325 |
| 24.481 | 3.6331 | 27 | 9 | 5.4 | 32 | 2 | 0.142 |
| 26.047 | 3.4182 | 25 | 38 | 22.8 | 312 | 19.7 | 0.349 |
| 27.996 | 3.1845 | 27 | 14 | 8.4 | 66 | 4.2 | 0.189 |
| 28.3 | 3.1509 | 28 | 7 | 4.2 | 14 | 0.9 | 0.1 |
| 29.028 | 3.0736 | 28 | 8 | 4.8 | 13 | 0.8 | 0.065 |
| 29.85 | 2.9907 | 26 | 5 | 3 | 10 | 0.6 | 0.1 |
| 30.955 | 2.8864 | 24 | 7 | 4.2 | 36 | 2.3 | 0.206 |
| 32.277 | 2.7712 | 23 | 18 | 10.8 | 59 | 3.7 | 0.131 |
| 33.605 | 2.6647 | 25 | 11 | 6.6 | 105 | 6.6 | 0.382 |
| 34.261 | 2.6152 | 24 | 11 | 6.6 | 105 | 6.6 | 0.382 |
| 39.225 | 2.2948 | 20 | 9 | 5.4 | 46 | 2.9 | 0.204 |
| 45.882 | 1.9762 | 16 | 8 | 4.8 | 27 | 1.7 | 0.135 |

TABLE 5
Peak Search Report (32 Peaks, Max P/N = 8.6
[Z00130.RAW] RQ9145 1476-10#3 2 3OKV-60MA 2-60DEG 2T

PEAK: 13-pts/Parabolic Filter, Threshold = 3.0, Cutoff = 0.1%,
BG = 3/1.0, Peak-Top = Summit

| 2-Theta | d(A) | BG | Height | 1% | Area | 1% | FWHM |
|---|---|---|---|---|---|---|---|
| 4.028 | 21.9196 | 22 | 319 | 100 | 2530 | 100 | 0.337 |
| 5.9 | 14.9672 | 18 | 4 | 1.3 | 8 | 0.3 | 0.1 |
| 9.164 | 9.642 | 11 | 11 | 3.4 | 64 | 2.5 | 0.233 |
| 12.207 | 7.2447 | 11 | 48 | 15 | 388 | 15.3 | 0.344 |
| 12.992 | 6.8084 | 17 | 16 | 5 | 86 | 3.4 | 0.215 |
| 14.351 | 6.1665 | 15 | 109 | 34.2 | 687 | 27.2 | 0.268 |
| 16.399 | 5.4008 | 15 | 58 | 18.2 | 579 | 22.9 | 0.424 |
| 18.003 | 4.9232 | 16 | 182 | 57.1 | 1804 | 71.3 | 0.421 |
| 19.121 | 4.6379 | 36 | 24 | 7.5 | 105 | 4.2 | 0.175 |
| 20.201 | 4.3921 | 24 | 215 | 67.4 | 1592 | 62.9 | 0.315 |
| 20.587 | 4.3106 | 23 | 55 | 17.2 | 689 | 27.2 | 0532 |
| 21.05 | 42169 | 26 | 2 | 0.6 | 4 | 0.2 | 0.1 |
| 21.563 | 4.1178 | 21 | 24 | 7.5 | 85 | 3.4 | 0.151 |
| 22.15 | 4.0099 | 19 | 4 | 1.3 | 8 | 0.3 | 0.1 |
| 22.902 | 3.8799 | 18 | 109 | 34.2 | 753 | 29.8 | 0.294 |
| 23.596 | 3.7673 | 19 | 40 | 12.5 | 271 | 10.7 | 0288 |
| 24.594 | 3.6166 | 17 | 16 | 5 | 64 | 2.5 | 0.16 |
| 25.402 | 3.5035 | 20 | 16 | 5 | 178 | 7 | 0.445 |
| 26.059 | 3.4166 | 22 | 78 | 24.5 | 610 | 2401 | 0.332 |
| 28.195 | 3.1625 | 24 | 28 | 8.8 | 210 | 8.3 | 0.319 |
| 29.228 | 3.053 | 24 | 29 | 9.1 | 195 | 7.7 | 0.286 |
| 31.098 | 2.8735 | 16 | 17 | 5.3 | 195 | 7.7 | 0.459 |
| 32.454 | 2.7565 | 15 | 45 | 14.1 | 299 | 11.8 | 0.282 |
| 33.551 | 2.6688 | 17 | 12 | 38 | 128 | 5.1 | 0.427 |
| 35.345 | 2.5373 | 18 | 8 | 2.5 | 22 | 0.9 | 0.11 |
| 37.276 | 2.4102 | 15 | 11 | 3.4 | 65 | 2.6 | 0.236 |
| 39.653 | 2.271 | 17 | 18 | 5.6 | 284 | 11.2 | 0.631 |
| 40.069 | 2.2484 | 20 | 16 | 5 | 171 | 6.8 | 0.428 |
| 41.037 | 2.1976 | 16 | 8 | 2.5 | 29 | 1.1 | 0.145 |
| 45.833 | 1.9782 | 12 | 10 | 3.1 | 117 | 4.6 | 0.468 |
| 50.772 | 1.7367 | 11 | 6 | 1.9 | 17 | 0.7 | 0.133 |
| 55.912 | 1.6431 | 9 | 6 | 1.9 | 17 | 0.7 | 0.113 |

While the invention has been described in detail and with reference to specific embodiments thereof, it will be apparent to one skilled in the art that various changes and modifications can be made therein without departing from the spirit and scope thereof.

What is claimed is:

1. A pharmaceutical composition comprising a pharmaceutically active agent and a salt of said pharmaceutically active agent with the proviso that said composition does not contain hydrolyzed cellulose, wherein said pharmaceutical active agent is a weak acid or weak base.

2. The pharmaceutical composition of claim 1 in at least one solid, non-crystalline state.

3. The pharmaceutical composition of claim 1 in at least one solid, crystalline state.

4. The pharmaceutical composition of claim 1 in at least one liquid crystalline form.

5. The pharmaceutical composition of claim 1 in at least one gel form.

6. A form of a pharmaceutically active agent prepared by reacting in an aqueous solvent a portion of said pharmaceutically active agent with an acid or base capable of reacting with said pharmaceutical active agent so as to form a mixture of said pharmaceutical active agent with its salt followed by drying, wherein said pharmaceutically active agent with a conjugate acid or base and said drying is conducted under conditions such that the pharmaceutically active agent is in intimate contact with its salt.

7. A form of a pharmaceutically active agent prepared by reacting a portion of said pharmaceutically active agent in molten form with an acid or base capable of reacting with said pharmaceutically active agent, wherein said pharmaceutically active agent is a weak acid or weak base and said form contains a counter ion in a molar amount that is different from the molar amount necessary to form a salt of said pharmaceutically active agent with a conjugate acid or base.

8. A form of pharmaceutically active agent prepared by processing the pharmaceutically active agent with a salt of its conjugate acid or conjugate base in the presence of heat, pressure, shear and/or water, wherein said form contains a counter ion of said salt in a molar amount that is different from the molar amount necessary to form a salt of said pharmaceutically active agent with a conjugate acid or base.

9. An ibuprofen form having, when potassium is present in said form as a counter ion, an IR peak at 1706 cm⁻¹ shifted and broadened in absorbance relative to racemic ibuprofen free acid, said form's characteristic absorbance profile from 1000 to 650 cm⁻¹ and D-spacings of 21.1, 7.1, and 3.4 Å by X-ray diffraction.

10. The ibuprofen form of claim 9, wherein said ibuprofen form has a melting point range of 118-124° C. at a purity of at least 98% using differential scanning calorimetry when potassium is present in said form as a counter ion.

11. The ibuprofen form of claim 9 made by a process comprising the steps of reacting a base in an aqueous solution with ibuprofen followed by drying.

12. The ibuprofen form of claim 9 made by a process comprising the step of reacting a base with molten ibuprofen.

13. The ibuprofen form of claim 9 prepared by processing the pharmaceutically active agent with a salt of its conjugate acid or conjugate base in the presence of heat. Pressure, shear and/or water.

14. A solid dosage form comprising the ibuprofen form of claim 9.

15. A process for making the ibuprofen form of 9 comprising the step of reacting a base in aqueous solution with ibuprofen followed by drying.

16. A process for making the ibuprofen form of claim 9 comprising the step of reacting a base in aqueous solution with ibuprofen followed by drying.

17. A process for making the ibuprofen form of claim 9 comprising the step of processing ibuprofen with a salt of its conjugate acid or conjugate base in the presence of heat, pressure, shear and/or water.

18. A process for altering the absorption rate of a pharmaceutically active agent comprising reacting a pharmaceutically active agent with an acid or a base capable of reacting with said pharmaceutically active agent in an aqueous solution followed by drying, wherein said pharmaceutically active agent is a weak acid or weak base.

19. A process for altering the absorption rate of a pharmaceutically active agent comprising reacting a pharmaceutically active agent in a molten state with an acid or base capable of reacting with said pharmaceutically active agent, wherein said pharmaceutically active agent is a weak acid or weak base.

20. A drug delivery platform comprising a pharmaceutical active agent and a salt of said pharmaceutical active agent, wherein said pharmaceutical active agent is a weak acid or weak base and wherein said drug delivery platform is buffered.

21. The drug delivery platform of claim 20 with the proviso that said drug delivery platform does not contain hydrolyzed cellulose.

22. The solid dosage form of claim 14, further comprising at least one of a pharmaceutically acceptable excipient, said pharmaceutically active agent and a salt of said pharmaceutically active agent.

23. A method for improving dissolution of a pharmaceutically active agent comprising combining said pharmaceutically active agent with a salt of said pharmaceutically active agent.

24. The form of claim 6, wherein said molar amount of the counter ion is less than the molar amount necessary to form a salt of said pharmaceutically active agent with a conjugate acid or base.

25. The form of claim 7, wherein said molar amount of the counter ion is less than the molar amount necessary to form a salt of said pharmaceutically active agent with a conjugate acid or base.

26. The form of claim 8, wherein said molar amount of the counter ion is less than the molar amount necessary to form a salt of said pharmaceutically active agent with a conjugate acid or base.

27. The pharmaceutical composition of claim 1 in an aqueous based medium.

28. A drug delivery platform comprising the pharmaceutical composition of claim 1.

29. A drug delivery platform comprising the form of claim 6.

30. A drug delivery platform comprising the form of claim 7.

31. A drug delivery platform comprising the form of claim 8.

32. A drug delivery platform comprising the ibuprofen form of claim 9.

33. A method for improving the bioavailability of ibuprofen by providing a dosage form containing the new ibuprofen form of the invention and administering such to a patient whereby the new ibuprofen form dissolves into ibuprofen in vivo.

34. A dosage form comprising the pharmaceutical composition of claim 1.

35. A form of a pharmaceutically active agent prepared by reacting in an aqueous solvent a portion of said pharmaceutically active agent with an acid or base capable of reacting with said pharmaceutical active agent so as to form a mixture of said pharmaceutical active agent with its salt followed by drying, wherein said pharmaceutically active agent is a weak acid or weak base, said form contains a counter ion in a molar amount that is different from a molar amount necessary to form a salt of said pharmaceutically active agent with a conjugate acid or base and said drying is conducted under conditions such that the pharmaceutically active agent is in intimate contact with its salt.
